SEQUENCE LISTING (5 sequences)

SEQ ID NO: 1 - protein, 107 aa, Artificial Sequence
  Humanized antibody light chain
ETTVTQSPSFLSASVGDRVTITCITTTDIDDDMNWFQQEPGKAPKLLISEGNILRPGVPSRFSSSGYGTDFTLTISKLQPEDFATYYCLQSDNLPFTFGQGTKLEIK

SEQ ID NO: 2 - protein, 107 aa, Artificial Sequence
  Humanized antibody light chain variant
ETTVTQSPSFLSASVGDRVTITCITTTDIKRNMNWFQQEPGKAPKLLISQGNILRPGVPSRFSSSGYGTDFTLTISKLQPEDFATYYCLQSRGLPFTFGQGTKLEIK

SEQ ID NO: 3 - protein, 11 aa, Artificial Sequence
  Humanized antibody variant CDR
ITTTDIKRNMN

SEQ ID NO: 4 - protein, 7 aa, Artificial Sequence
  humanized antibody light chain variant CDR
QGNILRP

SEQ ID NO: 5 - protein, 9 aa, Artificial Sequence
  humanized antibody light chain variant CDR
LQSRGLPFT

(19) United States
(12) Patent Application Publication
(10) Pub. No.: US 2011/0212088 A1
(43) Pub. Date: Sep. 1, 2011

(54) ANTI-PAF ANTIBODIES

(76) Inventors: Roger A. SABBADINI, Lakeside, CA (US); Jonathan Michael WOJCIAK, Encinitas, CA (US)

(21) Appl. No.: 13/035,821

(22) Filed: Feb. 25, 2011

Related U.S. Application Data
(60) Provisional application No. 61/339,127, filed on Feb. 26, 2010.

(51) Int. Cl. A61K 39/395; C07K 16/18; C07F 9/02; C07K 14/00; G01N 33/53; A61P 29/00; A61P 37/00

(52) U.S. Cl. 424/133.1; 530/388.24; 530/387.3; 424/145.1; 558/169; 530/409; 435/7.92

(57) ABSTRACT

Monoclonal antibodies to platelet activating factor (PAF) are described, along with methods for their production and use. Such antibodies can be formulated and used for therapeutic purposes, as well as for diagnosis and detection.

### RELATED APPLICATIONS

This patent application claims the benefit of and priority to U.S. provisional patent application Ser. No. 61/339,127, filed on 26 Feb., 2010, which is incorporated herein in its entirety.

### SEQUENCE LISTING

The instant application contains a Sequence Listing which has been submitted via EFS-Web and is hereby incorporated by reference in its entirety. Said ASCII copy, created on Feb. 25, 2011, is named LPT3310UT.txt, and is 2,974 bytes in size.

### BACKGROUND OF THE INVENTION

1. Field of the Invention

The present invention relates to anti-lipid antibodies, particularly antibodies to the bioactive lipid platelet activating factor (PAF) and methods of making and using such antibodies.

The following description includes information that may be useful in understanding the present invention. It is not an admission that any of the information provided herein, or any publication specifically or implicitly referenced herein, is prior art, or even particularly relevant, to the presently claimed invention.

2. Background.

Platelet Activating Factor (PAF)

Platelet activating factor (PAF, 1-O-alkyl-2-acetyl-sn-glycero-3-phosphocholine) is an inflammatory mediator whose levels in serum are substantially elevated in patients having an inflammatory disease or disorder, for example, anaphylactic shock [see Okamoto H, Kamatani N. N Engl J. Med. (2008) 358:1516]. It has an acetyl group, CH3COO—, at the sn-2 position of the glycerol backbone, along with the ether-linked alkanyl group at the sn-1 position as shown:

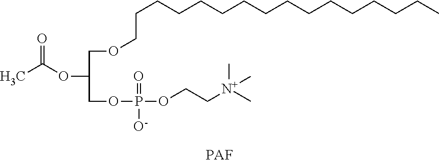

Having found that PAF was not sufficiently antigenic to allow production of PAF antibodies for use in immunoassays, Baldo (U.S. Pat. No. 5,061,626) developed a PAF analog (2-O-acetyl-1-0-(6′-oxohexyl)-sn-glyceryl-3-phosphorylcholine) that was conjugated to BSA and proved antigenic enough to immunize rabbits, yielding polyclonal anti-PAF antibodies. Polyclonal antibodies, however, are not well suited for therapeutic applications in human and non-human animals. Accordingly, there is a pressing need for monoclonal anti-PAF antibodies, which need is satisfied by this invention.

3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

The term “antibody” (“Ab”) or “immunoglobulin” (Ig) refers to any form of a peptide, polypeptide derived from, modeled after or encoded by, an immunoglobulin gene, or fragment thereof, that is capable of binding an antigen or epitope. See, e.g., IMMUNOBIOLOGY, Fifth Edition, C. A. Janeway, P. Travers, M., Walport, M. J. Shlomchiked., ed. Garland Publishing (2001). The term “antibody” is used herein in the broadest sense, and encompasses monoclonal, polyclonal or multispecific antibodies, minibodies, heteroconjugates, diabodies, triabodies, chimeric, antibodies, synthetic antibodies, antibody fragments, and binding agents that employ the complementarity determining regions (CDRs) of the parent antibody, or variants thereof that retain antigen binding activity. Antibodies are defined herein as retaining at least one desired activity of the parent antibody. Desired activities can include the ability to bind the antigen specifically, the ability to inhibit proleration in vitro, the ability to inhibit angiogenesis in vivo, and the ability to alter cytokine profile(s) in vitro.

Native antibodies (native immunoglobulins) are usually heterotetrameric glycoproteins of about 150,000 Daltons, typically composed of two identical light (L) chains and two identical heavy (H) chains. The heavy chain is approximately 50 kD in size, and the light chain is approximately 25 kDa. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The light chains of antibodies (immunoglobulins) from any vertebrate species can be assigned to one of two clearly distinct types, called kappa (κ) and lambda (λ), based on the amino acid sequences of their constant domains. The ratio of the two types of light chain varies from species to species. As a way of example, the average κ to λ, ratio is 20:1 in mice, whereas in humans it is 2:1 and in cattle it is 1:20.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An “antibody derivative” is an immune-derived moiety, i.e., a molecule that is derived from an antibody. This includes any antibody (Ab) or immunoglobulin (Ig), and refers to any form of a peptide, polypeptide derived from, modeled after or encoded by, an immunoglobulin gene, or a fragment of such peptide or polypeptide that is capable of binding an antigen or epitope. This comprehends, for example, antibody variants, antibody fragments, chimeric antibodies, humanized antibodies, multivalent antibodies, antibody conjugates and the like, which retain a desired level of binding activity for antigen.

As used herein, “antibody fragment” refers to a portion of an intact antibody that includes the antigen binding site or variable regions of an intact antibody, wherein the portion can be free of the constant heavy chain domains (e.g., CH2, CH3, and CH4) of the Fc region of the intact antibody. Alternatively, portions of the constant heavy chain domains (e.g., CH2, CH3, and CH4) can be included in the “antibody fragment”. Antibody fragments retain antigen-binding and include Fab, Fab′, F(ab′)₂, Fd, and Fv fragments; diabodies; triabodies; single-chain antibody molecules (sc-Fv); minibodies, nanobodies, and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. By way of example, a Fab fragment also contains the constant domain of a light chain and the first constant domain (CH1) of a heavy chain. “Fv” is the minimum antibody fragment that contains a complete antigen-recognition and -binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. “Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds. Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

An “antibody variant” refers herein to a molecule which differs in amino acid sequence from the amino acid sequence of a native or parent antibody that is directed to the same antigen by virtue of addition, deletion and/or substitution of one or more amino acid residue(s) in the antibody sequence and which retains at least one desired activity of the parent anti-binding antibody. Desired activities can include the ability to bind the parent antigen, retained or altered specificity for the parent antigen, and/or activity in one or more assays or models in vitro or in vivo. The variant will typically also have new desired activities such as ability to bind another antigen in addition to or in place of the parent antigen, enhanced stability, or enhanced pharmacokinetic or toxicological properties. The amino acid change(s) in an antibody variant may be within a variable region or a constant region of a light chain and/or a heavy chain, including in the Fc region, the Fab region, the CH₁ domain, the CH₂ domain, the CH₃ domain, and the hinge region. In one embodiment, the variant comprises one or more amino acid substitution(s) in one or more hypervariable region(s) of the parent antibody. For example, the variant may comprise at least one, e.g. from about one to about ten, and preferably from about two to about five, substitutions in one or more hypervariable regions of the parent antibody. Ordinarily, the variant will have an amino acid sequence having at least 50% amino acid sequence identity with the parent antibody heavy or light chain variable domain sequences, more preferably at least 65%, more preferably at 80%, more preferably at least 85%, more preferably at least 90%, and most preferably at least 95%. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind a bioactive lipid and preferably has desired activities which are superior to those of the parent antibody. For example, the variant may have a stronger binding affinity, different pharmacokinetic or toxicological properties, or enhanced ability to reduce angiogenesis and/or halt tumor progression. To analyze such desired properties (for example les immunogenic, longer half-life, enhanced stability, enhanced potency), one should compare a Fab form of the variant to a Fab form of the parent antibody or a full length form of the variant to a full length form of the parent antibody, for example, since it has been found that the format of the anti-sphingolipid antibody impacts its activity in the biological activity assays disclosed herein. The variant antibody of particular interest herein can be one which displays at least about 10 fold, preferably at least about % 5, 25, 59, or more of at least one desired activity. The preferred variant is one that has superior biophysical properties as measured in vitro or superior activities biological as measured in vitro or in vivo when compared to the parent antibody.

An “anti-PAF agent” refers to any therapeutic agent that binds PAF, and includes antibodies, antibody variants, antibody-derived molecules or non-antibody-derived moieties that bind PAF and its variants.

An “anti-PAF antibody” or an “immune-derived moiety reactive against PAF” refers to any antibody or antibody-derived molecule that binds PAF. As will be understood from these definitions, antibodies or immune-derived moieties may be polyclonal or monoclonal and may be generated through a variety of means, and/or may be isolated from an animal, including a human subject.

A “bioactive lipid” refers to a lipid signaling molecule. Bioactive lipids are distinguished from structural lipids (e.g., membrane-bound phospholipids) in that they mediate extracellular and/or intracellular signaling and thus are involved in controlling the function of many types of cells by modulating differentiation, migration, proliferation, secretion, survival, and other processes. In vivo, bioactive lipids can be found in extracellular fluids, where they can be complexed with other molecules, for example serum proteins such as albumin and lipoproteins, or in “free” form, i.e., not complexed with another molecule species. As extracellular mediators, some bioactive lipids alter cell signaling by activating membrane-bound ion channels or GPCRs or enzymes or factors that, in turn, activate complex signaling systems that result in changes in cell function or survival. As intracellular mediators, bioactive lipids can exert their actions by directly interacting with intracellular components such as enzymes, ion channels or structural elements such as actin. Examples of bioactive lipids include those characterized by a glycerol-based backbone, for example, platelet activating factor (PAF).

The term “biologically active,” in the context of an antibody or antibody fragment or variant, refers to an antibody or antibody fragment or antibody variant that is capable of binding the desired epitope and in some ways exerting a biologic effect. Biological effects include, but are not limited to, the modulation of a growth signal, the modulation of an anti-apoptotic signal, the modulation of an apoptotic signal, the modulation of the effector function cascade, and modulation of other ligand interactions.

A “biomarker” is a specific biochemical in the body which has a particular molecular feature that makes it useful for measuring the progress of disease or the effects of treatment. For example, S1P is a biomarker for certain hyperproliferative and/or cardiovascular conditions.

The term “cardiotherapeutic agent” refers to an agent that is therapeutic to diseases and diseases caused by or associated with cardiac and myocardial diseases and disorders.

“Cardiovascular therapy” encompasses cardiac therapy (treatment of myocardial ischemia and/or heart failure) as well as the prevention and/or treatment of other diseases associated with the cardiovascular system, such as heart disease. The term “heart disease” encompasses any type of disease, disorder, trauma or surgical treatment that involves the heart or myocardial tissue. Of particular interest are conditions associated with tissue remodeling. The term “cardiotherapeutic agent” refers to an agent that is therapeutic to diseases and diseases caused by or associated with cardiac and myocardial diseases and disorders.

A “carrier” refers to a moiety adapted for conjugation to a hapten, thereby rendering the hapten immunogenic. A representative, non-limiting class of carriers is proteins, examples of which include albumin, keyhole limpet hemocyanin, hemaglutanin, tetanus, and diptheria toxoid. Other classes and examples of carriers suitable for use in accordance with the invention are known in the art. These, as well as later discovered or invented naturally occurring or synthetic carriers, can be adapted for application in accordance with the invention.

As used herein, the expressions “cell,” “cell line,” and “cell culture” are used interchangeably and all such designations include progeny. Thus, the words “transformants” and “transformed cells” include the primary subject cell and cultures derived there from without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

“Cerebrovascular therapy” refers to therapy directed to the prevention and/or treatment of diseases and disorders associated with cerebral ischemia and/or hypoxia. Of particular interest is cerebral ischemia and/or hypoxia resulting from global ischemia resulting from a heart disease, including without limitation heart failure.

The term “chemotherapeutic agent” means anti-cancer and other anti-hyperproliferative agents. Thus chemotherapeutic agents are a subset of therapeutic agents in general. Chemotherapeutic agents include, but are not limited to: DNA damaging agents and agents that inhibit DNA synthesis: anthracyclines (doxorubicin, donorubicin, epirubicin), alkylating agents (bendamustine, busulfan, carboplatin, carmustine, chlorambucil, cyclophosphamide, dacarbazine, hexamethylmelamine, ifosphamide, lomustine, mechlorethamine, melphalan, mitotane, mytomycin, pipobroman, procarbazine, streptozocin, thiotepa, and triethylenemelamine), platinum derivatives (cisplatin, carboplatin, cis diammine-dichloroplatinum), and topoisomerase inhibitors (Camptosar); anti-metabolites such as capecitabine, chlorodeoxyadenosine, cytarabine (and its activated form, ara-CMP), cytosine arabinoside, dacabazine, floxuridine, fludarabine, 5-fluorouracil, 5-DFUR, gemcitabine, hydroxyurea, 6-mercaptopurine, methotrexate, pentostatin, trimetrexate, 6-thioguanine); anti-angiogenics (bevacizumab, thalidomide, sunitinib, lenalidomide, TNP-470, 2-methoxyestradiol, ranibizumab, sorafenib, erlotinib, bortezomib, pegaptanib, endostatin); vascular disrupting agents (flavonoids/flavones, DMXAA, combretastatin derivatives such as CA4DP, ZD6126, AVE8062A, etc.); biologics such as antibodies (Herceptin, Avastin, Panorex, Rituxin, Zevalin, Mylotarg, Campath, Bexxar, Erbitux); endocrine therapy: aromatase inhibitors (4-hydroandrostendione, exemestane, aminoglutehimide, anastrazole, letozole), anti-estrogens (Tamoxifen, Toremifine, Raoxifene, Faslodex), steroids such as dexamethasone; immuno-modulators: cytokines such as IFN-beta and IL2), inhibitors to integrins, other adhesion proteins and matrix metalloproteinases); histone deacetylase inhibitors like suberoylanilide hydroxamic acid; inhibitors of signal transduction such as inhibitors of tyrosine kinases like imatinib (Gleevec); inhibitors of heat shock proteins like 17-N-allylamino-17-demethoxygeldanamycin; retinoids such as all trans retinoic acid; inhibitors of growth factor receptors or the growth factors themselves; anti-mitotic compounds and/or tubulin-depolymerizing agents such as the taxoids (paclitaxel, docetaxel, taxotere, BAY 59-8862), navelbine, vinblastine, vincristine, vindesine and vinorelbine; anti-inflammatories such as COX inhibitors and cell cycle regulators, e.g., check point regulators and telomerase inhibitors.

The term “chimeric” antibody (or immunoglobulin) refers to a molecule comprising a heavy and/or light chain which is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly, et al., infra; Morrison et al., Proc. Natl. Acad. Sci. U.S.A., vol. 81:6851 (1984)).

The term “combination therapy” refers to a therapeutic regimen that involves the provision of at least two distinct therapies to achieve an indicated therapeutic effect. For example, a combination therapy may involve the administration of two or more chemically distinct active ingredients, for example, a fast-acting chemotherapeutic agent and an anti-lipid antibody, or two different antibodies. Alternatively, a combination therapy may involve the administration of an anti-lipid antibody together with the delivery of another treatment, such as radiation therapy and/or surgery. Further, a combination therapy may involve administration of an anti-lipid antibody together with one or more other biological agents (e.g., anti-VEGF, TGFβ, PDGF, or bFGF agent), chemotherapeutic agents and another treatment such as radiation and/or surgery. In the context of the administration of two or more chemically distinct active ingredients, it is understood that the active ingredients may be administered as part of the same composition or as different compositions. When administered as separate compositions, the compositions comprising the different active ingredients may be administered at the same or different times, by the same or different routes, using the same of different dosing regimens, all as the particular context requires and as determined by the attending physician. Similarly, when one or more anti-lipid antibody species, for example, an anti-LPA antibody, alone or in conjunction with one or more chemotherapeutic agents are combined with, for example, radiation and/or surgery, the drug(s) may be delivered before or after surgery or radiation treatment.

The term “constant domain” refers to the C-terminal region of an antibody heavy or light chain. Generally, the constant domains are not directly involved in the binding properties of an antibody molecule to an antigen, but exhibit various effector functions, such as participation of the antibody in antibody-dependent cellular toxicity. Here, “effector functions” refer to the different physiological effects of antibodies (e.g., opsonization, cell lysis, mast cell, basophil and eosinophil degranulation, and other processes) mediated by the recruitment of immune cells by the molecular interaction between the Fc domain and proteins of the immune system. The isotype of the heavy chain determines the functional properties of the antibody. Their distinctive functional properties are conferred by the carboxy-terminal portions of the heavy chains, where they are not associated with light chains.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

A “device” as used herein refers to an instrument, apparatus, implement, machine, appliance, implant, in vitro reagent or calibrator, software, matrix, plate, dipstick, column, material or other similar or related article which is intended to be used for one or more of the specific purpose(s) of diagnosis, prevention, monitoring, treatment or alleviation of disease or injury, or for providing information for medical or diagnostic purposes by means of ex vivo or in vitro examination of specimens derived from the human body.

A “derivatized bioactive lipid” is a bioactive lipid, e.g., PAF, which has a polar head group and at least one hydrocarbon chain, wherein a carbon atom within the hydrocarbon chain is derivatized with a reactive group [e.g., a sulfhydryl (thiol) group, a carboxylic acid group, a cyano group, an ester, a hydroxy group, an alkene, an alkyne, an acid chloride group or a halogen atom] that may or may not be protected. This derivatization serves to activate the bioactive lipid for reaction with a molecule, e.g., for conjugation to a carrier.

A “derivatized bioactive lipid conjugate” refers to a derivatized bioactive lipid that is covalently conjugated to a carrier. The carrier may be a protein molecule such as BSA or may be a non-proteinaceous moiety such as polyethylene glycol, colloidal gold, adjuvants or silicone beads. A derivatized bioactive lipid conjugate may be used as an immunogen for generating an antibody response according to the instant invention, and the same or a different bioactive lipid conjugate may be used as a detection reagent for detecting the antibody thus produced. In some embodiments the derivatized bioactive lipid conjugate is attached to a solid support when used for detection.

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

“Effective concentration” refers to the absolute, relative, and/or available concentration and/or activity, for example of certain undesired bioactive lipids. In other words, the effective concentration of a bioactive lipid is the amount of lipid available, and able, to perform its biological function in a given milieu. In the present invention, an immune-derived moiety such as, for example, a monoclonal antibody directed to a bioactive lipid such as PAF is able to reduce the effective concentration of the lipid by binding to the lipid and rendering it unable to perform its biological function. In this example, the lipid itself is still present (it is not degraded by the antibody, in other words) but can no longer bind its receptor or other targets to cause a downstream effect, so “effective concentration” rather than absolute concentration is the appropriate measurement. Methods and assays exist for directly and/or indirectly measuring the effective concentration of bioactive lipids.

An “epitope” or “antigenic determinant” refers to that portion of an antigen that reacts with an antibody antigen-binding portion derived from an antibody.

The term “expression cassette” refers to a nucleotide molecule capable of affecting expression of a structural gene (i.e., a protein coding sequence, such as an antibody of the invention) in a host compatible with such sequences. Expression cassettes include at least a promoter operably linked with the polypeptide-coding sequence, and, optionally, with other sequences, e.g., transcription termination signals. Additional regulatory elements necessary or helpful in effecting expression may also be used, e.g., enhancers. Thus, expression cassettes include plasmids, expression vectors, recombinant viruses, any form of recombinant “naked DNA” vector, and the like.

A “fully human antibody” can refer to an antibody produced in a genetically engineered (i.e., transgenic) mouse (e.g. from Medarex) that, when presented with an immunogen, can produce a human antibody that does not necessarily require CDR grafting. These antibodies are fully human (100% human protein sequences) from animals such as mice in which the non-human antibody genes are suppressed and replaced with human antibody gene expression. The applicants believe that antibodies could be generated against bioactive lipids when presented to these genetically engineered mice or other animals who might be able to produce human frameworks for the relevant CDRs.

A “hapten” is a substance that is non-immunogenic but can react with an antibody or antigen-binding portion derived from an antibody. In other words, haptens have the property of antigenicity but not immunogenicity. A hapten is generally a small molecule that can, under most circumstances, elicit an immune response (i.e., act as an antigen) only when attached to a carrier, for example, a protein, polyethylene glycol (PEG), colloidal gold, silicone beads, or the like. The carrier may be one that also does not elicit an immune response by itself. A representative, non-limiting class of hapten molecules is proteins, examples of which include albumin, keyhole limpet hemocyanin, hemaglutanin, tetanus, and diphtheria toxoid. Other classes and examples of hapten molecules are known in the art. These, as well as later discovered or invented naturally occurring or synthetic haptens, can be adapted for application in accordance with the invention.

The term “heteroconjugate antibody” can refer to two covalently joined antibodies. Such antibodies can be prepared using known methods in synthetic protein chemistry, including using crosslinking agents. As used herein, the term “conjugate” refers to molecules formed by the covalent attachment of one or more antibody fragment(s) or binding moieties to one or more polymer molecule(s).

“Humanized” forms of non-human (e.g., murine) antibodies are chimeric antibodies that contain minimal sequence derived from non-human immunoglobulin. Or, looked at another way, a humanized antibody is a human antibody that also contains selected sequences from non-human (e.g., murine) antibodies in place of the human sequences. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. In some instances, framework region (FR) residues of the human immunoglobulin are replaced by corresponding non-human residues.

Furthermore, humanized antibodies can comprise residues that are found neither in the recipient antibody nor in the imported CDR or framework sequences. These modifications are made to further refine and maximize antibody performance. Thus, in general, a humanized antibody will comprise all of at least one, and in one aspect two, variable domains, in which all or all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), or that of a human immunoglobulin. See, e.g., Cabilly, et al., U.S. Pat. No. 4,816,567; Cabilly, et al., European Patent No. 0,125,023 B1; Boss, et al., U.S. Pat. No. 4,816,397; Boss, et al., European Patent No. 0,120,694 B1; Neuberger, et al., WO 86/01533; Neuberger, et al., European Patent No. 0,194,276 B1; Winter, U.S. Pat. No. 5,225,539; Winter, European Patent No. 0,239,400 B1; Padlan, et al., European Patent Application No. 0,519,596 A1; Queen, et al. (1989), Proc. Nat'l Acad. Sci. USA, vol. 86:10029-10033). For further details, see Jones et al., Nature 321:522-525 (1986); Reichmann et al., Nature 332:323-329 (1988); and Presta, Curr. Op. Struct. Biol. 2:593-596 (1992) and Hansen, WO2006105062.

The term “hyperproliferative disorder” refers to diseases and disorders associated with, the uncontrolled proliferation of cells, including but not limited to uncontrolled growth of organ and tissue cells resulting in cancers and benign tumors. Hyperproliferative disorders associated with endothelial cells can result in diseases of angiogenesis such as angiomas, endometriosis, obesity, age-related macular degeneration and various retinopathies, as well as the proliferation of endothelial cells and smooth muscle cells that cause restenosis as a consequence of stenting in the treatment of atherosclerosis. Hyperproliferative disorders involving fibroblasts (i.e., fibrogenesis) include but are not limited to disorders of excessive scarring (i.e., fibrosis) such as age-related macular degeneration, cardiac remodeling and failure associated with myocardial infarction, excessive wound healing such as commonly occurs as a consequence of surgery or injury, keloids, and fibroid tumors and stenting.

An “immune-derived moiety” includes any antibody (Ab) or immunoglobulin (Ig), and refers to any form of a peptide, polypeptide derived from, modeled after or encoded by, an immunoglobulin gene, or a fragment of such peptide or polypeptide that is capable of binding an antigen or epitope (see, e.g., Immunobiology, 5th Edition, Janeway, Travers, Walport, Shlomchiked. (editors), Garland Publishing (2001)). In the present invention, the antigen is a lipid molecule, such as a bioactive lipid molecule.

An “immunogen” is a molecule capable of inducing a specific immune response, particularly an antibody response in an animal to whom the immunogen has been administered. In the instant invention, the immunogen is a derivatized bioactive lipid conjugated to a carrier, i.e., a “derivatized bioactive lipid conjugate”. The derivatized bioactive lipid conjugate used as the immunogen may be used as capture material for detection of the antibody generated in response to the immunogen. Thus the immunogen may also be used as a detection reagent. Alternatively, the derivatized bioactive lipid conjugate used as capture material may have a different linker and/or carrier moiety from that in the immunogen.

The phrase “in silico” refers to computer simulations that model natural or laboratory processes.

To “inhibit,” particularly in the context of a biological phenomenon, means to decrease, suppress or delay. For example, a treatment yielding “inhibition of tumorigenesis” may mean that tumors do not form at all, or that they form more slowly, or are fewer in number than in the untreated control.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition, such as one that is conjugated directly or indirectly to the antibody. The label may itself be detectable by itself (e.g., radioisotope labels or fluorescent labels) or, in the case of an enzymatic label, may catalyze chemical alteration of a substrate compound or composition that is detectable.

A “ligand” is a substance that is able to bind to and form a complex with a biomolecule to serve a biological purpose. Thus an antigen may be described as a ligand of the antibody to which it binds.

A “liposome” is a small vesicle composed of various types of lipids, phospholipids and/or surfactant that is useful for delivery of a drug (such as the anti-sphingolipid antibodies disclosed herein and, optionally, a chemotherapeutic agent) to a mammal. The components of the liposome are commonly arranged in a bilayer formation, similar to the lipid arrangement of biological membranes. An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

In the context of this invention, a “liquid composition” refers to one that, in its filled and finished form as provided from a manufacturer to an end user (e.g., a doctor or nurse), is a liquid or solution, as opposed to a solid. Here, “solid” refers to compositions that are not liquids or solutions. For example, solids include dried compositions prepared by lyophilization, freeze-drying, precipitation, and similar procedures.

The expression “linear antibodies” when used throughout this application refers to the antibodies described in Zapata et al. Protein Eng. 8(10):1057-1062 (1995). Briefly, these antibodies comprise a pair of tandem Fd segments (V_{H}-C_{H}1-V_{H}-C_{H}1) that form a pair of antigen binding regions. Linear antibodies can be bispecific or monospecific.

The term “metabolites” refers to compounds from which PAF is made, as well as those that result from the degradation of PAF; that is, compounds that are involved in the PAF metabolic pathways. The term “metabolic precursors” may be used to refer to compounds from which sphingolipids are made.

The term “monoclonal antibody” (mAb) as used herein refers to an antibody obtained from a population of substantially homogeneous antibodies, or to said population of antibodies. The individual antibodies comprising the population are essentially identical, except for possible naturally occurring mutations and/or post-translational modifications that may occur present during cell culture or antibody production. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567) which may or may not involve in silico design steps as described herein. The “monoclonal antibodies” may also be isolated from phage antibody libraries using the techniques described in Clackson et al., Nature 352:624-628 (1991) and Marks et al., J. Mol. Biol. 222:581-597 (1991), for example, or by other methods known in the art. The monoclonal antibodies herein specifically include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Monotherapy” refers to a treatment regimen based on the delivery of one therapeutically effective compound, whether administered as a single dose or several doses over time.

The term “multispecific antibody” can refer to an antibody, or a monoclonal antibody, having binding properties for at least two different epitopes. In one embodiment, the epitopes are from the same antigen. In another embodiment, the epitopes are from two or more different antigens. Methods for making multispecific antibodies are known in the art. Multispecific antibodies include bispecific antibodies (having binding properties for two epitopes), trispecific antibodies (three epitopes) and so on. For example, multispecific antibodies can be produced recombinantly using the co-expression of two or more immunoglobulin heavy chain/light chain pairs. Alternatively, multispecific antibodies can be prepared using chemical linkage. One of skill can produce multispecific antibodies using these or other methods as may be known in the art. Multispecific antibodies include multispecific antibody fragments. One example of a multispecific (in this case, bispecific) antibody comprehended by this invention is an antibody having binding properties for an S1P epitope and a C1P epitope, which thus is able to recognize and bind to both S1P and C1P. Another example of a bispecific antibody comprehended by this invention is an antibody having binding properties for an epitope from a bioactive lipid and an epitope from a cell surface antigen. Thus the antibody is able to recognize and bind the bioactive lipid and is able to recognize and bind to cells, e.g., for targeting purposes.

“Neoplasia” or “cancer” refers to abnormal and uncontrolled cell growth. A “neoplasm”, or tumor or cancer, is an abnormal, unregulated, and disorganized proliferation of cell growth, and is generally referred to as cancer. A neoplasm may be benign or malignant. A neoplasm is malignant, or cancerous, if it has properties of destructive growth, invasiveness, and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The “parent” antibody herein is one that is encoded by an amino acid sequence used for the preparation of the variant. The parent antibody may be a native antibody or may already be a variant, e.g., a chimeric antibody. For example, the parent antibody may be a humanized or human antibody.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the non-patentable embodiment(s). Also, the claims appended hereto are to be interpreted both to provide the broadest reasonable scope, as well as to preserve their validity. Furthermore, the claims are to be interpreted in a way that (1) preserves their validity and (2) provides the broadest reasonable interpretation under the circumstances, if one or more of the statutory requirements for patentability are amended or if the standards change for assessing whether a particular statutory requirement for patentability is satisfied from the time this application is filed or issues as a patent to a time the validity of one or more of the appended claims is questioned.

The term “pharmaceutically acceptable salt” refers to a salt, such as used in formulation, which retains the biological effectiveness and properties of the agents and compounds of this invention and which are is biologically or otherwise undesirable. In many cases, the agents and compounds of this invention are capable of forming acid and/or base salts by virtue of the presence of charged groups, for example, charged amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts (see Berge, et al. (1977) J. Pharm. Sci., vol. 66, 1-19).

A “plurality” means more than one.

The term “promoter” includes all sequences capable of driving transcription of a coding sequence in a cell. Thus, promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. Transcriptional regulatory regions suitable for use in the present invention include but are not limited to the human cytomegalovirus (CMV) immediate-early enhancer/promoter, the SV40 early enhancer/promoter, the E. coli lac or trp promoters, and other promoters known to control expression of genes in prokaryotic or eukaryotic cells or their viruses.

The term “recombinant DNA” refers to nucleic acids and gene products expressed therefrom that have been engineered, created, or modified by man. “Recombinant” polypeptides or proteins are polypeptides or proteins produced by recombinant DNA techniques, for example, from cells transformed by an exogenous DNA construct encoding the desired polypeptide or protein. “Synthetic” polypeptides or proteins are those prepared by chemical synthesis.

The terms “separated”, “purified”, “isolated”, and the like mean that one or more components of a sample contained in a sample-holding vessel are or have been physically removed from, or diluted in the presence of, one or more other sample components present in the vessel. Sample components that may be removed or diluted during a separating or purifying step include, chemical reaction products, non-reacted chemicals, proteins, carbohydrates, lipids, and unbound molecules.

By “solid phase” is meant a non-aqueous matrix such as one to which the antibody of the present invention can adhere. Examples of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles, such as those described in U.S. Pat. No. 4,275,149.

The term “species” is used herein in various contexts, e.g., a particular species of chemotherapeutic agent. In each context, the term refers to a population of chemically indistinct molecules of the sort referred in the particular context.

The term “specific” or “specificity” in the context of antibody-antigen interactions refers to the selective, non-random interaction between an antibody and its target epitope. Here, the term “antigen” refers to a molecule that is recognized and bound by an antibody molecule or other immune-derived moiety. The specific portion of an antigen that is bound by an antibody is termed the “epitope”. This interaction depends on the presence of structural, hydrophobic/hydrophilic, and/or electrostatic features that allow appropriate chemical or molecular interactions between the molecules. Thus an antibody is commonly said to “bind” (or “specifically bind”) or be “reactive with” (or “specifically reactive with”), or, equivalently, “reactive against” (or “specifically reactive against”) the epitope of its target antigen. Antibodies are commonly described in the art as being “against” or “to” their antigens as shorthand for antibody binding to the antigen. Thus an “antibody that binds PAF,” an “antibody that specifically binds PAF,” an “antibody reactive against PAF,” an “antibody reactive with PAF,” an “antibody to PAF” and an “anti-PAF antibody” all have the same meaning in the art. Antibody molecules can be tested for specificity of binding by comparing binding to the desired antigen to binding to unrelated antigen or analogue antigen or antigen mixture under a given set of conditions. Preferably, an antibody according to the invention will lack significant binding to unrelated antigens, or even analogs of the target antigen. “Specifically associate” and “specific association” and the like refer to a specific, non-random interaction between two molecules, which interaction depends on the presence of structural, hydrophobic/hydrophilic, and/or electrostatic features that allow appropriate chemical or molecular interactions between the molecules.

Herein, “stable” refers to an interaction between two molecules (e.g., a peptide and a TLR molecule) that is sufficiently stable such that the molecules can be maintained for the desired purpose or manipulation. For example, a “stable” interaction between a peptide and a TLR molecule refers to one wherein the peptide becomes and remains associated with a TLR molecule for a period sufficient to achieve the desired effect.

A “subject” or “patient” refers to an animal in need of treatment that can be effected by molecules of the invention. Animals that can be treated in accordance with the invention include vertebrates, with mammals such as bovine, canine, equine, feline, ovine, porcine, and primate (including humans and non-human primates) animals being particularly preferred examples.

A “surrogate marker” refers to laboratory measurement of biological activity within the body that indirectly indicates the effect of treatment on disease state.

A “therapeutic agent” refers to a drug or compound that is intended to provide a therapeutic effect including, but not limited to: anti-inflammatory drugs including COX inhibitors and other NSAIDS, anti-angiogenic drugs, chemotherapeutic drugs as defined above, cardiovascular agents, immunomodulatory agents, agents that are used to treat neurodegenerative disorders, opthalmic drugs, anti-fibrotics, etc.

A “therapeutically effective amount” (or “effective amount”) refers to an amount of an active ingredient, e.g., an agent according to the invention, sufficient to effect treatment when administered to a subject in need of such treatment. Accordingly, what constitutes a therapeutically effective amount of a composition according to the invention may be readily determined by one of ordinary skill in the art. In the context of cancer therapy, a “therapeutically effective amount” is one that produces an objectively measured change in one or more parameters associated with cancer cell survival or metabolism, including an increase or decrease in the expression of one or more genes correlated with the particular cancer, reduction in tumor burden, cancer cell lysis, the detection of one or more cancer cell death markers in a biological sample (e.g., a biopsy and an aliquot of a bodily fluid such as whole blood, plasma, serum, urine, etc.), induction of induction apoptosis or other cell death pathways, etc. Of course, the therapeutically effective amount will vary depending upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of the active ingredient when administered as a monotherapy (i.e., a therapeutic regimen that employs only one chemical entity as the active ingredient).

The compositions of the invention are used in methods of bioactive lipid-based therapy. As used herein, the terms “therapy” and “therapeutic” encompasses the full spectrum of prevention and/or treatments for a disease, disorder or physical trauma. A “therapeutic” agent of the invention may act in a manner that is prophylactic or preventive, including those that incorporate procedures designed to target individuals that can be identified as being at risk (pharmacogenetics); or in a manner that is ameliorative or curative in nature; or may act to slow the rate or extent of the progression of at least one symptom of a disease or disorder being treated; or may act to minimize the time required, the occurrence or extent of any discomfort or pain, or physical limitations associated with recuperation from a disease, disorder or physical trauma; or may be used as an adjuvant to other therapies and treatments. The term “treatment” or “treating” means any treatment of a disease or disorder, including preventing or protecting against the disease or disorder (that is, causing the clinical symptoms not to develop); inhibiting the disease or disorder (i.e., arresting, delaying or suppressing the development of clinical symptoms; and/or relieving the disease or disorder (i.e., causing the regression of clinical symptoms). As will be appreciated, it is not always possible to distinguish between “preventing” and “suppressing” a disease or disorder because the ultimate inductive event or events may be unknown or latent. Those “in need of treatment” include those already with the disorder as well as those in which the disorder is to be prevented. Accordingly, the term “prophylaxis” will be understood to constitute a type of “treatment” that encompasses both “preventing” and “suppressing”. The term “protection” thus includes “prophylaxis”.

The term “therapeutic regimen” means any treatment of a disease or disorder using chemotherapeutic and cytotoxic agents, radiation therapy, surgery, gene therapy, DNA vaccines and therapy, siRNA therapy, anti-angiogenic therapy, immunotherapy, bone marrow transplants, aptamers and other biologics such as antibodies and antibody variants, receptor decoys and other protein-based therapeutics.

The “variable” region of an antibody comprises framework and complementarity determining regions (CDRs, otherwise known as hypervariable regions). The variability is not evenly distributed throughout the variable domains of antibodies. It is concentrated in six CDR segments, three in each of the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases forming part of, the beta-sheet structure. The term “hypervariable region” when used herein refers to the amino acid residues of an antibody which are responsible for antigen binding. The hypervariable region comprises amino acid residues from a “complementarity determining region” or “CDR” (for example residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (for example residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

A “vector” or “plasmid” or “expression vector” refers to a nucleic acid that can be maintained transiently or stably in a cell to effect expression of one or more recombinant genes. A vector can comprise nucleic acid, alone or complexed with other compounds. A vector optionally comprises viral or bacterial nucleic acids and/or proteins, and/or membranes. Vectors include, but are not limited, to replicons (e.g., RNA replicons, bacteriophages) to which fragments of DNA may be attached and become replicated. Thus, vectors include, but are not limited to, RNA, autonomous self-replicating circular or linear DNA or RNA and include both the expression and non-expression plasmids. Plasmids can be commercially available, publicly available on an unrestricted basis, or can be constructed from available plasmids as reported with published protocols. In addition, the expression vectors may also contain a gene to provide a phenotypic trait for selection of transformed host cells such as dihydrofolate reductase or neomycin resistance for eukaryotic cell culture, or such as tetracycline or ampicillin resistance in E. coli.

### SUMMARY OF THE INVENTION

Antibodies reactive with platelet activating factor (PAF) are described, as are compositions that include such antibodies. Various methods for making and using such antibodies, as well as other PAF-binding moieiies derived from such antibodies, are also provided.

One aspect of the invention concerns isolated monoclonal antibodies, or antigen binding fragments thereof, that bind PAF. Particularly preferred embodiments include isolated humanized anti-PAF antibodies that comprise two heavy chains and two light chains, as well as various antigen binding fragments thereof, e.g., Fab fragments. In some embodiments, the anti-PAF antibody or antigen binding fragment thereof has at least one amino acid residue that is glycosylated. Such antibodies, or PAF-binding fragments of such antibodies, can be formulated into any suitable composition. For therapeutic applications, pharmaceutical or veterinary compositions comprising such isolated anti-PAF antibodies or antigen binding fragments and an acceptable carrier, for example, a pharmaceutically or veterinarily acceptable carrier, are preferred.

Preferably, anti-PAF antibodies and antigen binding fragments of the invention include at least one heavy chain variable region and at least one light chain variable region. Preferably each heavy and light chain variable region comprises at least three CDRs. In the context light chain variable regions, they preferably include at least one, and preferably two and/or three, of the following light chain CDRs: a CDRL1 comprising the amino acid sequence ITTTDIKRNMN (SEQ ID NO: 3) or a variant thereof in which from 1 to 10 amino acids residues are substituted with a different amino acid residue; a CDRL2 comprising the amino acid sequence QGNILRP (SEQ ID NO: 4) or a variant thereof in which from 1 to 6 amino acids residues are substituted with a different amino acid residue; and/or a CDRL3 comprising the amino acid sequence LQSRGLPFT (SEQ ID NO: 5) or a variant thereof in which from 1 to 8 amino acids residues are substituted with a different amino acid residue.

In certain preferred embodiments of this aspect, the light chain variable domain of the anti-PAF antibody or antigen binding fragment thereof comprises a sequence of amino acid residues having the following amino acid sequence: ETTVTQSPSFLSASVGDRVTITCITTTDIKRNMNWFQQEPGKAPKLLISQGNILRPGVPSRFSS SGYGTDFTLTISKLQPEDFATYYCLQSRGLPFTFGQGTKLEIK (SEQ ID NO: 2), or a sequence of amino acid residues that has an amino acid sequence that has at least 50%, 65%, 80%, 85%, 90%, or 95% sequence identity with such amino acid sequence.

Yet another aspect concerns methods of making the anti-PAF antibodies and antigen binding fragments of the invention. Typically, these molecules are produced by recombinant expression, whereby nucleic acids encoding polypeptides having the desired amino acid sequences are stably introduced into and then expressed (either constitutively or inducibly, in suitable host cells such as mammalian cell lines.

Still other aspects of the invention relate to methods of using the anti-PAF antibodies and antigen binding fragments of the invention. One such aspect involves methods of reducing inflammation, allergic responses, or immune responses in a subject. Such methods include administering to a subject having undesired inflammation or an undesired allergic or immune response a therapeutically effective amount of an isolated antibody, or an antigen binding fragment thereof, according to the invention. Another such aspect relates to methods of treating a disease or condition in a subject, comprising administering to a subject a therapeutically effective amount of an isolated antibody, or an antigen binding fragment thereof, according to claim 1, wherein the disease or condition is an inflammatory diseases or condition, a disease or condition having an inflammatory component, an autoimmune disease or condition, an allergic condition, inflammatory bowel disease, ulcerative colitis, Crohn's disease, spondyloarthropathy, osteoarthritis, rheumatoid arthritis, multiple sclerosis, immune suppression, systemic lupus erythematosis, psoriasis, asthma, glomerulonephritis, thyroiditis, chondrocalcinosis, acute lung injury, sepsis, ischemia-reperfusion injury, acute respiratory distress syndrome, neuropathic pain, hydrostatic pulmonary edema or trauma.

Another aspect of the invention concerns diagnostic reagents that include a derivatized PAF molecule having a polar head group and a hydrocarbon chain attached to the polar head group that has a carbon atom within the hydrocarbon chain derivatized with a reactive group. The derivatized carbon atom preferably is located within a portion the hydrocarbon chain that is not adjacent to the polar head group, and is preferably the distal or terminal carbon atom of the hydrocarbon chain. A particularly preferred reactive group is a sulfhydryl (thiol) group. The reactive group facilitates direct or indirect association or conjugation, covalently or non-covalently, of derivatized PAF molecules with other molecules, moieties, or structures, including solid supports (e.g., plastic beads or plates, materials used to form column matrices, etc.) or carrier moieties, for example, polyethylene glycol, colloidal gold, adjuvant, a silicone bead, and a protein, wherein the protein is optionally selected from the group consisting of keyhole limpet hemocyanin, albumin, ovalbumin, bovine thyroglobulin, and soybean trypsin inhibitor.

The diagnostic reagents of the invention have many uses, including in ELISA kits. Such kits can include, for example, a diagnostic reagent according to the invention and an agent that binds PAF under physiological conditions (e.g., an anti-PAF antibody or antigen binding fragment according to the invention). They can also be used to detect whether a biological sample contains a PAF binding agent (e.g., an anti-PAF antibody, antibody fragment, or antibody derivative according to the invention, a PAF receptor, and autoantibody to PAF, etc.). Such assays are typically performed under conditions that allow the diagnostic reagent to bind the PAF binding agent, if present in the sample. In some preferred embodiments the diagnostic reagent is labeled, for example, with biotin, a fluorescent reporter, a radionuclide, or other detectable label substance. Alternatively, the diagnostic reagents of the invention can also be used to detect (quantitatively, semi-quantitatively, or qualitatively) if a sample, particularly a biological sample, contains PAF or a metabolite thereof. Such methods generally involve performing an assay to detect PAF (or a PAF metabolite) binding, usually through the use of an anti-PAF antibody or antigen binding fragment according to the invention under conditions that allow the anti-PAF antibody (or PAF-binding fragment) to bind to PAF (or PAF metabolite) molecules if present in the sample. Biological samples that can be diagnostically assayed include tissue samples (e.g., a biopsy sample) as well as fluid samples such whole blood, plasma, serum, urine, semen, bile, aqueous humor, vitreous humor, synovial fluid, bronchioalveolar lavage fluid, mucous, and sputum.

These and other aspects and embodiments of the invention are discussed in greater detail in the sections that follow. The foregoing and other aspects of the invention will become more apparent from the following detailed description, accompanying drawings, and the claims. Although methods and materials similar or equivalent to those described herein can be used in the practice or testing of the present invention, suitable methods and materials are described below. In addition, the materials, methods, and examples below are illustrative only and not intended to be limiting.

### BRIEF DESCRIPTION OF THE DRAWINGS

This application contains at least one figure executed in color. Copies of this application with color drawing(s) will be provided upon request and payment of the necessary fee. A brief summary of each of the figures is provided below.

FIG. 1 is a line graph showing binding of PAF antibodies (from hybridomas 9B7, 6C5 and 15B3) to plates coated with a PAF conjugate (FIG. 1A) or with a lysophosphatidic acid (LPA) conjugate (FIG. 1B). As can be seen from the graphs, all three antibodies bind well to PAF but not to the closely related lipid LPA.

FIG. 2 is a line graph showing saturation binding of PAF-biotin to a PAF antibody isolated from hybridoma 9B7. The EC50 for binding is shown to be approximately 200 nM.

FIG. 3 is a line graph showing inhibition of native PAF by antibody 9B7 in vitro, using the DiscoveRx PAF receptor signaling assay.

### DETAILED DESCRIPTION OF THE INVENTION

1. Antibody Compounds.

Antibody molecules or immunoglobulins are large glycoprotein molecules with a molecular weight of approximately 150 kDa, usually composed of two different kinds of polypeptide chain. The heavy chain (H) is approximately 50 kDa. The light chain (L), is approximately 25 kDa. Each immunoglobulin molecule usually consists of two heavy chains and two light chains. The two heavy chains are linked to each other by disulfide bonds, the number of which varies between the heavy chains of different immunoglobulin isotypes. Each light chain is linked to a heavy chain by one covalent disulfide bond. In any given naturally occurring antibody molecule, the two heavy chains and the two light chains are identical, harboring two identical antigen-binding sites, and are thus said to be divalent, i.e., having the capacity to bind simultaneously to two identical molecules.

The light chains of antibody molecules from any vertebrate species can be assigned to one of two clearly distinct types, kappa (k) and lambda (l), based on the amino acid sequences of their constant domains. The ratio of the two types of light chain varies from species to species. As a way of example, the average k to l ratio is 20:1 in mice, whereas in humans it is 2:1 and in cattle it is 1:20.

The heavy chains of antibody molecules from any vertebrate species can be assigned to one of five clearly distinct types, called isotypes, based on the amino acid sequences of their constant domains. Some isotypes have several subtypes. The five major classes of immunoglobulin are immunoglobulin M (IgM), immunoglobulin D (IgD), immunoglobulin G (IgG), immunoglobulin A (IgA), and immunoglobulin E (IgE). IgG is the most abundant isotype and has several subclasses (IgG1, 2, 3, and 4 in humans). The Fc fragment and hinge regions differ in antibodies of different isotypes, thus determining their functional properties. However, the overall organization of the domains is similar in all isotypes.

Sources of antibody are not limited to those exemplified herein (e.g., murine and humanized murine antibody). Antibodies may be raised in many species including mammalian species (for example, mouse, rat, camel, bovine, goat, horse, guinea pig, hamster, sheep and rabbit) and birds (duck, chicken). Antibodies raised may derive from a different species from the animal in which they are raised. For example, the XenoMouse™ (Abgenix, Inc., Fremont Calif.) produces fully human monoclonal antibodies. For certain purposes, native human antibodies, such as autoantibodies to PAF isolated from individuals who may show a titer of such PAF autoantibody may be used. Alternatively, a human antibody sequence library may be used to generate antibodies comprising a human sequence.

2. Antibody Applications.

Therapeutic agents that alter the activity or concentration of one or more undesired bioactive lipids, or precursors or metabolites thereof, are therapeutically useful. These agents, including antibodies, act by changing the effective concentration, i.e., the absolute, relative, effective and/or available concentration and/or activities, of certain undesired bioactive lipids, in a given milieu. Lowering the effective concentration of the bioactive lipid may be said to “neutralize” the target lipid or its undesired effects, including downstream effects. Here, “undesired” refers to a bioactive lipid that is unwanted due to its involvement in a disease process, for example, as a signaling molecule, or to an unwanted amount of a bioactive lipid which contributes to disease when present in excess.

Without wishing to be bound by any particular theory, it is believed that inappropriate concentrations of bioactive lipids, such as PAF and/or its metabolites or downstream effectors, may cause or contribute to the development of various diseases and disorders, including inflammatory diseases and disorders. As such, the compositions and methods can be used to treat these diseases and disorders, particularly by decreasing the effective in vivo concentration of PAF.

One way to control the amount of undesirable PAF in a patient is by providing a composition that comprises one or more humanized anti-PAF antibodies to bind PAF, thereby acting as therapeutic “sponges” that reduce the level of free undesirable PAF. When a compound is referred to as “free”, the compound is not in any way restricted from reaching the site or sites where it exerts its undesirable effects. Typically, a free compound is present in blood and tissue, which either is or contains the site(s) of action of the free compound, or from which a compound can freely migrate to its site(s) of action. A free compound may also be available to be acted upon by any enzyme that converts the compound into an undesirable compound. Without wishing to be bound by any particular theory, it is believed that an undesirable level of PAF causes or contributes to the development of various inflammatory diseases and disorders, among others.

Such humanized anti-sphingolipid antibodies may be formulated in a pharmaceutical composition and are useful for a variety of purposes, including the treatment of diseases, disorders or physical trauma. Pharmaceutical compositions comprising one or more humanized anti-PAF antibodies of the invention may be incorporated into kits and medical devices for such treatment. Medical devices may be used to administer the pharmaceutical compositions of the invention to a patient in need thereof, and according to one embodiment of the invention, kits are provided that include such devices. Such devices and kits may be designed for routine administration, including self-administration, of the pharmaceutical compositions of the invention. Such devices and kits may also be designed for emergency use, for example, in ambulances or emergency rooms, or during surgery, or in activities where injury is possible but where full medical attention may not be immediately forthcoming (for example, hiking and camping, or combat situations).

Methods of Administration.

The treatment for diseases and conditions discussed herein can be achieved by administering agents and compositions of the invention by various routes employing different formulations and devices. Suitable pharmaceutically acceptable diluents, carriers, and excipients are well known in the art. One skilled in the art will appreciate that the amounts to be administered for any particular treatment protocol can readily be determined. Suitable amounts might be expected to fall within the range of 10 μg/dose to 10 g/dose, preferably within 10 mg/dose to 1 g/dose.

Drug substances may be administered by techniques known in the art, including but not limited to systemic, subcutaneous, intradermal, mucosal, including by inhalation, and topical administration. The mucosa refers to the epithelial tissue that lines the internal cavities of the body. For example, the mucosa comprises the alimentary canal, including the mouth, esophagus, stomach, intestines, and anus; the respiratory tract, including the nasal passages, trachea, bronchi, and lungs; and the genitalia. For the purpose of this specification, the mucosa also includes the external surface of the eye, i.e., the cornea and conjunctiva. Local administration (as opposed to systemic administration) may be advantageous because this approach can limit potential systemic side effects, but still allow therapeutic effect.

Pharmaceutical compositions used in the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations used in the present invention may be prepared according to conventional techniques well known in the pharmaceutical industry. Such techniques include the step of bringing into association the active ingredients with the pharmaceutical carrier(s) or excipient(s). Preferred carriers include those that are pharmaceutically acceptable, particularly when the composition is intended for therapeutic use in humans. For non-human therapeutic applications (e.g., in the treatment of companion animals, livestock, fish, or poultry), veterinarily acceptable carriers may be employed. In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment the pharmaceutical compositions may be formulated and used as foams. Pharmaceutical foams include formulations such as, but not limited to, emulsions, microemulsions, creams, jellies, and liposomes.

While basically similar in nature these formulations vary in the components and the consistency of the final product. The know-how on the preparation of such compositions and formulations is generally known to those skilled in the pharmaceutical and formulation arts and may be applied to the formulation of the compositions of the present invention.

Various excipients might also be added to the formulated antibody to improve performance of the therapy, make the therapy more convenient or to clearly ensure that the formulated antibody is used only for its intended, approved purpose. Examples of excipients include chemicals to control pH, antimicrobial agents, preservatives to prevent loss of antibody potency, solubilizing agents to increase the concentration of antibody in the formulation, penetration enhancers and the use of agents to adjust isotonicity and/or viscosity. Inhibitors of, e.g., proteases, could be added to prolong the half life of the antibody.

The anti-bioactive lipid agent (e.g., a humanized antibody) can also be chemically modified to yield a pro-drug that is administered in one of the formulations or devices previously described above. The active form of the antibody is then released by action of an endogenous enzyme. Possible ocular enzymes to be considered in this application are the various cytochrome p450s, aldehyde reductases, ketone reductases, esterases or N-acetyl-β-glucosamidases. Other chemical modifications to the antibody could increase its molecular weight, and as a result, increase the residence time of the antibody in a particular tissue or compartment. An example of such a chemical modification is pegylation [Harris and Chess (2003), Nat Rev Drug Discov; 2: 214-21], a process that can be general or specific for a functional group such as disulfide [Shaunak, et al. (2006), Nat Chem Biol; 2:312-3] or a thiol [Doherty, et al. (2005), Bioconjug Chem; 16: 1291-8].

Antibody Characterization

Antibody affinities may be determined, e.g., as described in the examples herein below. Preferred humanized or variant antibodies are those which bind PAF with a K_{d} value of no more than about 1×10⁻⁷ M, preferably no more than about 1×10⁻⁸ M, and most preferably no more than about 5×10⁻⁹ M.

The term half maximal effective concentration (EC50) refers to the concentration of antibody which induces a response halfway between the baseline and maximum. Preferably, the EC50 for binding of the antibody to PAF is less than about 1 micromolar, more preferably less than about 500 nM, more preferably less than about 250 nM, including less than about 200 nM. In other embodiments the EC50 for binding of the antibody to PAF is less than about 100 nM, less than about 50 nM, less than about 20 nM, less than about 10 nM or less than about 5 nM. In one embodiment these values are obtained in an ELISA assay. In one embodiment the ELISA assay is a “reverse” ELISA, in which antibody binding to PAF (optionally biotinylated PAF) is measured.

Preferably, the antibody has an effective concentration 50 (EC50) value of no more than about 10 ug/ml, preferably no more than about 1 ug/ml, and most preferably no more than about 0.1 ug/ml, e.g. as measured in a direct binding ELISA assay. Preferably, the antibody has an effective concentration value of no more than about 10 ug/ml, preferably no more than about 1 ug/ml, and most preferably no more than about 0.1 ug/ml, as measured in cell assays. Preferably, the antibody has an effective concentration value of no more than about 10 ug/ml, preferably no more than about 1 ug/ml, and most preferably no more than about 0.1 ug/ml.

Assays for determining the activity of the anti-PAF antibodies of the invention include ELISA assays, optionally direct binding ELISAs or “reverse” ELISAs.

Aside from antibodies with strong binding affinity for PAF, it is also desirable to select humanized or variant antibodies that have other beneficial properties from a therapeutic perspective. For example, the antibody may be one that reduce inflammation in anaphylaxis.

Preferably the humanized or variant anti-antibody fails to elicit an immunogenic response upon administration of a therapeutically effective amount of the antibody to a human patient. If an immunogenic response is elicited, preferably the response will be such that the antibody still provides a therapeutic benefit to the patient treated therewith.

According to one embodiment of the invention, humanized anti-PAF antibodies bind the “epitope” as herein defined. To screen for antibodies that bind to an epitope on PAF, a routine cross-blocking assay such as that described in Antibodies, A Laboratory Manual, Cold Spring Harbor Laboratory, Ed Harlow and David Lane (1988), can be performed. Alternatively, epitope mapping, e.g., as described in Champe, et al. [J. Biol. Chem. 270:1388-1394 (1995)], can be performed to determine whether the antibody binds an epitope of interest.

Conventional Antibody Generation and Characterization

The antibodies of the invention have a heavy chain variable domain comprising an amino acid sequence represented by the formula: FR1-CDRH1-FR2-CDRH2-FR3-CDRH3-FR4, wherein “FR1-4” represents the four framework regions and “CDRH1-3” represents the three hypervariable regions of an anti-sphingolipid antibody variable heavy domain. FR1-4 may be derived from a “consensus sequence” (for example the most common amino acids of a class, subclass or subgroup of heavy or light chains of human immunoglobulins) as in the examples below or may be derived from an individual human antibody framework region or from a combination of different framework region sequences. Many human antibody framework region sequences are compiled in Kabat, et al., supra, for example. In one embodiment, the variable heavy FR is provided by a consensus sequence of a human immunoglobulin subgroup as compiled by Kabat, et al., above.

The human variable heavy FR sequence preferably has one or more substitutions therein, e.g., wherein the human FR residue is replaced by a corresponding nonhuman residue (by “corresponding nonhuman residue” is meant the nonhuman residue with the same Kabat positional numbering as the human residue of interest when the human and nonhuman sequences are aligned), but replacement with the nonhuman residue is not necessary. For example, a replacement FR residue other than the corresponding nonhuman residue can be selected by phage display. Exemplary variable heavy FR residues which may be substituted include any one or more of FR residue numbers: 37H, 49H, 67H, 69H, 71H, 73H, 75H, 76H, 78H, and 94H (Kabat residue numbering employed here). Preferably at least two, or at least three, or at least four of these residues are substituted. A particularly preferred combination of FR substitutions is: 49H, 69H, 71H, 73H, 76H, 78H, and 94H. With respect to the heavy chain hypervariable regions, these preferably have amino acid sequences listed in Table 2, below.

The antibodies of the preferred embodiment herein have a light chain variable domain comprising an amino acid sequence represented by the formula: FR1-CDRL1-FR2-CDRL2-FR3-CDRL3-FR4, wherein “FR1-4” represents the four framework regions and “CDRL1-3” represents the three hypervariable regions of an anti-sphingolipid antibody variable heavy domain. FR1-4 may be derived from a “consensus sequence” (for example, the most common amino acids of a class, subclass or subgroup of heavy or light chains of human immunoglobulins) as in the examples below or may be derived from an individual human antibody framework region or from a combination of different framework region sequences. In one preferred embodiment, the variable light FR is provided by a consensus sequence of a human immunoglobulin subgroup as compiled by Kabat, et al., above.

The human variable light FR sequence preferably has substitutions therein, e.g., wherein a human FR residue is replaced by a corresponding mouse residue, but replacement with the nonhuman residue is not necessary. For example, a replacement residue other than the corresponding nonhuman residue may be selected by phage display.

Methods for generating humanized anti-sphingolipid antibodies of interest herein are elaborated in more detail below.

### A. Antibody Preparation

Methods for preparing anti-PAF antibodies are described in the Examples below and in commonly owned, co-pending U.S. patent application Ser. Nos. 12/258,337, 12/258,383, 12/690,033, 12/418,597, 12/129,109, 12/406,874, 61/170,595, and 12/660,528, each of which is hereby incorporated by reference in its entirety for any and all purposes.

(i) Antigen Preparation.

The PAF antigen to be used for production of antibodies may be intact PAF. In one embodiment, PAF is derivatized, and may be associated with a carrier protein. See, e.g., commonly owned, co-pending U.S. patent application Ser. Nos. 11/755,352 and 11/755,699, each of which is hereby incorporated by reference in its entirety for any and all purposes.

(ii) Polyclonal Antibodies.

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 ug or 5 ug of the protein or conjugate (for rabbits or mice, respectively) with three volumes of Freund's complete adjuvant and injecting the solution intradermally at multiple sites. One month later the animals are boosted with 0.1 to 0.2 times the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum may be suitably used to enhance the immune response.

(iii) Monoclonal Antibodies.

Monoclonal antibodies may be made using the hybridoma method first described by Kohler, et al., Nature, 256:495 (1975), or by other suitable methods, including by recombinant DNA methods (see, e.g., U.S. Pat. No. 4,816,567). In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOP-21 and M.C.-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur, et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbant assay (ELISA).

The binding affinity of a monoclonal antibody can, for example, be determined by the Scatchard analysis of Munson, et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

(iv) Humanization and Amino Acid Sequence Variants.

General methods for antibody humanization are described in, for example, U.S. Pat. No. 5,861,155, US19960652558, U.S. Pat. No. 6,479,284, US20000660169, U.S. Pat. No. 6,407,213, US19930146206, U.S. Pat. No. 6,639,055, US20000705686, U.S. Pat. No. 6,500,931, US19950435516, U.S. Pat. No. 5,530,101, U.S. Pat. No. 5,585,089, US19950477728, U.S. Pat. No. 5,693,761, US19950474040, U.S. Pat. No. 5,693,762, US19950487200, U.S. Pat. No. 6,180,370, US19950484537, US2003229208, US20030389155, U.S. Pat. No. 5,714,350, US19950372262, U.S. Pat. No. 6,350,861, US19970862871, U.S. Pat. No. 5,777,085, US19950458516, U.S. Pat. No. 5,834,597, US19960656586, U.S. Pat. No. 5,882,644, US19960621751, U.S. Pat. No. 5,932,448, US19910801798, US6013256, US19970934841, U.S. Pat. No. 6,129,914, US19950397411, U.S. Pat. No. 6,210,671, U.S. Pat. No. 6,329,511, US19990450520, US2003166871, US20020078757, U.S. Pat. No. 5,225,539, US19910782717, U.S. Pat. No. 6,548,640, US19950452462, U.S. Pat. No. 5,624,821, and US19950479752. In certain embodiments, it may be desirable to generate amino acid sequence variants of these humanized antibodies, particularly where these improve the binding affinity or other biological properties of the humanized antibody. Examples hereinbelow describe methodologies for generating amino acid sequence variants of an anti-sphingolipid antibody with enhanced affinity relative to the parent antibody.

Amino acid sequence variants of a parent anti-PAF antibody are prepared by introducing appropriate nucleotide changes into the anti-sphingolipid antibody DNA, or by peptide synthesis. Such variants include, for example, deletions from, and/or insertions into and/or substitutions of, residues within the amino acid sequences of the anti-sphingolipid antibodies of the examples herein. Any combination of deletion, insertion, and substitution is made to arrive at the final construct, provided that the final construct possesses the desired characteristics. The amino acid changes also may alter post-translational processes of the humanized or variant anti-PAF antibody, such as changing the number or position of glycosylation sites.

One type of variant is an amino acid substitution variant. These variants have at least one amino acid residue in the anti-PAF antibody molecule removed and a different residue inserted in its place. The sites of greatest interest for substitutional mutagenesis include the hypervariable regions, but FR alterations are also contemplated. Conservative substitutions are preferred substitutions. If such substitutions result in a change in biological activity, then more substantial changes, denominated “exemplary” substitutions listed below, or as further described below in reference to amino acid classes, may be introduced and the products screened.

TABLE 1
Exemplary Amino Acid Residue Substitutions

| Amino acid residue (symbol) | Exemplary substitutions |
|---|---|
| Ala (A) | val; leu; ile val |
| Arg (R) | lys; gln; asn lys |
| Asn (N) | gln; his; asp, lys; gln arg |
| Asp (D) | glu; asn glu |
| Cys (C) | ser; ala ser |
| Gln (Q) | asn; glu asn |
| Glu (E) | asp; gln asp |
| Gly (G) | ala ala |
| His (H) | asn; gln; lys; arg arg |
| Ile (I) | leu; val; met; ala; leu phe; norleucine |
| Leu (L) | norleucine; ile; val; ile met; ala; phe |
| Lys (K) | arg; gln; asn arg |
| Met (M) | leu; phe; ile leu |
| Phe (F) | leu; val; ile; ala; tyr tyr |
| Pro (P) | ala ala |
| Ser (S) | thr thr |
| Thr (T) | ser ser |
| Trp (W) | tyr; phe tyr |
| Tyr (Y) | trp; phe; thr; ser phe |
| Val (V) | ile; leu; met; phe; leu ala; norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class.

Any cysteine residue not involved in maintaining the proper conformation of the humanized or variant anti-PAF antibody also may be substituted, to improve the oxidative stability of the molecule and prevent aberrant crosslinking. Conversely, cysteine bond(s) may be added to the antibody to improve its stability (particularly where the antibody is an antibody fragment such as an Fv fragment).

One type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants is affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene IIII product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or in addition, it may be beneficial to analyze a crystal structure of the antigen-antibody complex to identify contact points between the antibody and sphingolipid. Such contact residues and neighboring residues are candidates for substitution according to the techniques elaborated herein. Crystals (co-crystals) of the antigen—antibody complex include co-crystals of the antigen and the Fab or other fragment of the antibody, along with any salts, metals (including divalent metals), cofactors and the like. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked and/or or O-linked. N-linked refers to the attachment of the carbohydrate moiety to the side chain of an asparagine residue. The tripeptide sequences asparagine-X-serine and asparagine-X-threonine, where X is any amino acid except proline, are the most common recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-sphingolipid antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-sphingolipid antibody.

(v) Human Antibodies.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits, et al., Proc. Natl. Acad. Sci. USA, 90:2551 (1993); Jakobovits, et al., Nature, 362:255-258 (1993); Bruggermann, et al., Year in Immuno., 7:33 (1993); and U.S. Pat. Nos. 5,591,669, 5,589,369 and 5,545,807. Human antibodies can also be derived from phage-display libraries (Hoogenboom, et al., J. Mol. Biol., 227:381 (1991); Marks, et al., J. Mol. Biol., 222:581-597 (1991); and U.S. Pat. Nos. 5,565,332 and 5,573,905). As discussed above, human antibodies may also be generated by in vitro activated B cells (see, e.g., U.S. Pat. Nos. 5,567,610 and 5,229,275) or by other suitable methods.

(vi) Antibody Fragments.

In certain embodiments, the humanized or variant anti-PAF antibody is an antibody fragment. Various techniques have been developed for the production of antibody fragments. Traditionally, these fragments were derived via proteolytic digestion of intact antibodies (see, e.g., Morimoto, et al., Journal of Biochemical and Biophysical Methods 24:107-117 (1992); and Brennan, et al., Science 229:81 (1985)). However, these fragments can now be produced directly by recombinant host cells. For example, Fab′-SH fragments can be directly recovered from E. coli and chemically coupled to form F(ab′)₂ fragments (Carter, et al., Bio/Technology 10:163-167 (1992)). In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture. Other techniques for the production of antibody fragments will be apparent to the skilled practitioner.

(vii) Multispecific Antibodies.

In some embodiments, it may be desirable to generate multispecific (e.g., bispecific) humanized or variant anti-PAF antibodies having binding specificities for at least two different epitopes. Exemplary bispecific antibodies may bind to two different epitopes of PAF. Alternatively, an anti-PAF binding region may be combined with a region which binds to a different molecule, e.g., a bioactive lipid such as a sphingolipid (e.g., S1P) or a lysophosphatidic acid (LPA). Bispecific antibodies can be prepared as full length antibodies or antibody fragments (e.g., F(ab′)₂ bispecific antibodies).

According to another approach for making bispecific antibodies, the interface between a pair of antibody molecules can be engineered to maximize the percentage of heterodimers that are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. See, e.g., U.S. Pat. No. 5,731,168.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods. Suitable cross-linking agents are well known in the art, and are disclosed in, for example, U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan, et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes. In yet a further embodiment, Fab′-SH fragments directly recovered from E. coli can be chemically coupled in vitro to form bispecific antibodies. Shalaby, et al., J. Exp. Med. 175:217-225 (1992).

Various techniques for making and isolating bispecific antibody fragments directly from recombinant cell culture have also been described. For example, bispecific antibodies have been produced using leucine zippers. Kostelny, et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger, et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, e.g., Gruber, et al., J. Immunol. 152:5368 (1994). Alternatively, the bispecific antibody may be a “linear antibody” produced as described in, fror example, Zapata, et al. Protein Eng. 8(10):1057-1062 (1995).

Antibodies with more than two valencies are also contemplated. For example, trispecific antibodies can be prepared. Tutt et al., J. Immunol. 147:60 (1991).

An antibody (or polymer or polypeptide) of the invention comprising one or more binding sites per arm or fragment thereof will be referred to herein as “multivalent” antibody. For example a “bivalent” antibody of the invention comprises two binding sites per Fab or fragment thereof whereas a “trivalent” polypeptide of the invention comprises three binding sites per Fab or fragment thereof. In a multivalent polymer of the invention, the two or more binding sites per Fab may be binding to the same or different antigens. For example, the two or more binding sites in a multivalent polypeptide of the invention may be directed against the same antigen, for example against the same parts or epitopes of said antigen or against two or more same or different parts or epitopes of said antigen; and/or may be directed against different antigens; or a combination thereof. Thus, a bivalent polypeptide of the invention for example may comprise two identical binding sites, may comprise a first binding sites directed against a first part or epitope of an antigen and a second binding site directed against the same part or epitope of said antigen or against another part or epitope of said antigen; or may comprise a first binding sites directed against a first part or epitope of an antigen and a second binding site directed against the a different antigen. However, as will be clear from the description hereinabove, the invention is not limited thereto, in the sense that a multivalent polypeptide of the invention may comprise any number of binding sites directed against the same or different antigens.

An antibody (or polymer or polypeptide) of the invention that contains at least two binding sites per Fab or fragment thereof, in which at least one binding site is directed against a first antigen and a second binding site directed against a second antigen different from the first antigen, will also be referred to as “multispecific”. Thus, a “bispecific” polymer comprises at least one site directed against a first antigen and at least one a second site directed against a second antigen, whereas a “trispecific” is a polymer that comprises at least one binding site directed against a first antigen, at least one further binding site directed against a second antigen, and at least one further binding site directed against a third antigen, etc. Accordingly, in their simplest form, a bispecific polypeptide of the invention is a bivalent polypeptide (per Fab) of the invention. However, as will be clear from the description hereinabove, the invention is not limited thereto, in the sense that a multispecific polypeptide of the invention may comprise any number of binding sites directed against two or more different antigens.

(viii) Other Modifications.

Other modifications of the humanized or variant anti-sphingolipid antibody are contemplated. For example, the invention also pertains to immunoconjugates comprising the antibody described herein conjugated to a cytotoxic agent such as a toxin (e.g., an enzymatically active toxin of bacterial, fungal, plant or animal origin, or fragments thereof), or a radioactive isotope (for example, a radioconjugate). Conjugates are made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

The anti-PAF antibodies disclosed herein may also be formulated as immunoliposomes. Liposomes containing the antibody are prepared by methods known in the art, such as described in Epstein et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. For example, liposomes can be generated by the reverse phase evaporation method with a lipid composition comprising phosphatidyl choline, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin, et al., J. Biol. Chem. 257:286-288 (1982) via a disulfide interchange reaction. Another active ingredient is optionally contained within the liposome.

Enzymes or other polypeptides can be covalently bound to the anti-PAF antibodies by techniques well known in the art such as the use of the heterobifunctional crosslinking reagents discussed above. Alternatively, fusion proteins comprising at least the antigen binding region of an antibody of the invention linked to at least a functionally active portion of an enzyme of the invention can be constructed using recombinant DNA techniques well known in the art (see, e.g., Neuberger, et al., Nature 312:604-608 (1984)).

It may be desirable to use an antibody fragment, rather than an intact antibody, to increase penetration of target tissues and cells, for example. In this case, it may be desirable to modify the antibody fragment in order to increase its serum half life. This may be achieved, for example, by incorporation of a salvage receptor binding epitope into the antibody fragment (e.g., by mutation of the appropriate region in the antibody fragment or by incorporating the epitope into a peptide tag that is then fused to the antibody fragment at either end or in the middle, e.g., by DNA or peptide synthesis). See, e.g., U.S. Pat. No. 6,096,871.

Covalent modifications of the humanized or variant anti-PAF antibody are also included within the scope of this invention. They may be made by chemical synthesis or by enzymatic or chemical cleavage of the antibody, if applicable. Other types of covalent modifications of the antibody are introduced into the molecule by reacting targeted amino acid residues of the antibody with an organic derivatizing agent that is capable of reacting with selected side chains or the N- or C-terminal residues. Exemplary covalent modifications of polypeptides are described in U.S. Pat. No. 5,534,615, specifically incorporated herein by reference. A preferred type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

B. Vectors, Host Cells and Recombinant Methods

The invention also provides isolated nucleic acid encoding the humanized or variant anti-PAF antibody, vectors and host cells comprising the nucleic acid, and recombinant techniques for the production of the antibody.

For recombinant production of the antibody, the nucleic acid encoding it may be isolated and inserted into a replicable vector for further cloning (amplification of the DNA) or for expression. In another embodiment, the antibody may be produced by homologous recombination, e.g., as described in U.S. Pat. No. 5,204,244. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence, as described, for example, in U.S. Pat. No. 5,534,615.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for anti-PAF antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated anti-PAF antibodies are preferably derived from multicellularorganisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham, et al., J. Gen Virol. 36:59 (1977)); baby hamster kidney cells (BHK, ATCC CCL 10); Chinese hamster ovary cells/−DHFR (CHO, Urlaub, et al., Proc. Natl. Acad. Sci. USA 77:4216 (1980)); mouse Sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather, et al., Annals N.Y. Acad. Sci. 383:44-68 (1982)); MRC 5 cells; FS4 cells; a human hepatoma line (Hep G2); and the PER.C6® cell line (Crucell).

Host cells are transformed with the above-described expression or cloning vectors for anti-PAF antibody production and cultured in conventional nutrient media modified as appropriate for inducing promoters, selecting transformants, or amplifying the genes encoding the desired sequences.

The host cells used to produce the anti-PAF antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham, et al., Meth. Enz. 58:44 (1979), Barnes, et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter, et al., Bio/Technology 10:163-167 (1992) describe a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human heavy chains (Lindmark, et al., J. Immunol. Meth. 62:1-13 (1983)). Protein G is recommended for all mouse isotypes and for human γ3 (Guss, et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the Bakerbond ABX™ resin (J. T. Baker, Phillipsburg, N.J.) is useful for purification. Other techniques for protein purification, such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

C. Pharmaceutical Formulations

Therapeutic formulations of an antibody or immune-derived moiety of the invention are prepared for storage by mixing the antibody having the desired degree of purity with optional physiologically acceptable carriers, excipients, or stabilizers (see, e.g., Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

The formulations to be used for in vivo administration must be sterile. This is readily accomplished for instance by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

D. Non-therapeutic Uses for the Antibodies

Antibodies to bioactive lipids may be used as affinity purification agents. In this process, the antibodies are immobilized on a solid phase such a Sephadex resin or filter paper, using methods well known in the art. The immobilized antibody is contacted with a sample containing the sphingolipid to be purified, and thereafter the support is washed with a suitable solvent that will remove substantially all the material in the sample except the sphingolipid, which is bound to the immobilized antibody. Finally, the support is washed with another suitable solvent, such as glycine buffer, for instance between pH 3 to pH 5.0, that will release the sphingolipid from the antibody.

Anti-lipid antibodies may also be useful in diagnostic assays for the target lipid, e.g., detecting its expression in specific cells, tissues (such as biopsy samples), or bodily fluids. Such diagnostic methods may be useful in diagnosis of inflammatory diseases or disorders.

The antibodies of the present invention may be employed in any known assay method, such as competitive binding assays, direct and indirect sandwich assays, and immunoprecipitation assays. See, e.g., Zola, Monoclonal Antibodies: A Manual of Techniques, pp. 147-158 (CRC Press, Inc. 1987).

Competitive binding assays rely on the ability of a labeled standard to compete with the test sample analyte for binding with a limited amount of antibody. The amount of bioactive lipid in the test sample is inversely proportional to the amount of standard that becomes bound to the antibodies. To facilitate determining the amount of standard that becomes bound, the antibodies generally are insoluble before or after the competition, so that the standard and analyte that are bound to the antibodies may conveniently be separated from the standard and analyte that remain unbound.

Sandwich assays involve the use of two antibodies, each capable of binding to a different immunogenic portion, or epitope, of the protein to be detected. In a sandwich assay, the test sample analyte is bound by a first antibody that is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the blood or tissue sample may be fresh or frozen or may be embedded in paraffin and fixed with a preservative such as formalin, for example.

The antibodies may also be used for in vivo diagnostic assays. Generally, the antibody is labeled with a radionuclide (such as ¹¹¹In, ⁹⁹Tc, ¹⁴C, ¹³¹I, ¹²⁵I, ³²P, or ³⁵S) so that the bound target molecule can be localized using immunoscintillography.

E. Compositions and Kits for Diagnosis and Detection

Antibodies to PAF, generally along with derivatized PAF, may be used to measure PAF in a biological sample, which may be for purposes of diagnosing diseases associated with PAF, or for providing information about PAF levels in a biological sample. This information may be useful in understanding and/or treating PAF-associated diseases and conditions, as well as diseases, injuries and conditions for which PAF is a biomarker. For example, circulating (serum) PAF levels are significantly elevated in human patients with anaphylaxis, and correlate with the severity of the anaphylactic symptoms. Vadas et al (2008) New Engl. J. Med. 358:28-35.

The biological sample in which PAF is detected may be a tissue sample, e.g., a biopsy sample, or a bodily fluid sample. Biological fluid samples include whole blood, plasma, serum, urine, semen, bile, aqueous humor, vitreous humor, synovial fluid, mucus, bronchioalveolar lavage fluid, and sputum.

As a matter of convenience, antibodies to bioactive lipids, or derivatized bioactive lipids, or both, as desired, can be provided in a kit, for example, a packaged combination of reagents in predetermined amounts with instructions for performing a diagnostic or detection assay. Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

In one embodiment, a direct ELISA kit is provided which contains a PAF conjugate (e.g., PAF-SMCC-BSA, PAF-IOA-Ovalbumin or other PAF conjugate) and an antibody to PAF. Optionally the kit contains one or more plates or other solid supports, and optionally the plates or solid supports are pre-coated with PAF or PAF conjugate. Optionally the kit may also contain a PAF standard, and may contain other solutions or reagents needed to run the assay.

In another embodiment, a PAF-binding ELISA or inverted PAF ELISA kit is provided which contains labeled PAF, such as biotinylated PAF, an antibody to PAF, and an antibody for detecting the PAF antibody. In one embodiment, the inverted ELISA format, the murine anti-PAF antibody is captured on the plate coated with an anti-mouse antibody. Labeled lipid (e.g., biotinylated PAF) is then titrated and allowed to bind the immobilized anti-PAF antibody. The labeled lipid is then detected, for example using an HRP-streptavidin secondary to detect the biotinylated lipid.

Optionally the kit contains one or more plates or other solid supports, and optionally the plates or solid supports are pre-coated with the secondary antibody. Optionally the kit may also contain detection reagents for the label (e.g. HRP—conjugated streptavidin in the case of biotinylated PAF), and may contain other solutions or reagents needed to run the assay.

F. Therapeutic Uses for the Antibody

For therapeutic applications, antibodies to PAF are administered to a mammal, preferably a human, in a pharmaceutically acceptable dosage form such as those discussed above, including those that may be administered to a human intravenously as a bolus or by continuous infusion over a period of time, by intramuscular, intraperitoneal, intra-cerebrospinal, subcutaneous, intra-articular, intrasynovial, intrathecal, oral, topical, or inhalation routes, as the particular therapeutic regimen requires.

For the prevention or treatment of disease, the appropriate dosage of antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 ug/kg to about 50 mg/kg (e.g., 0.1-20 mg/kg) of antibody is an initial candidate dosage for administration to the patient, whether, for example, by one or more separate administrations, or by continuous infusion. A typical daily or weekly dosage might range from about 1 μg/kg to about 20 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic imaging.

According to another embodiment of the invention, the effectiveness of the antibody in preventing or treating disease may be improved by administering the antibody serially or in combination with another agent that is effective for those purposes, such as chemotherapeutic anti-cancer drugs, for example. Such other agents may be present in the composition being administered or may be administered separately. The antibody is suitably administered serially or in combination with the other agent.

It is believed that decreasing the effective concentration of PAF, as can be accomplished using the antibodies and methods of the invention, will be therapeutically useful in the treatment and/or prevention of inflammatory diseases or diseases with an inflammatory component. PAF has been implicated in a range of autoimmune and allergic conditions, including inflammatory bowel disease, ulcerative colitis, Crohn's disease, spondyloarthropathies, osteoarthritis, rheumatoid arthritis, multiple sclerosis, immune suppression, systemic lupus erythematosis, psoriasis, asthma, glomerulonephritis, thyroiditis [Edwards and Constantinescu, 2009, Inflammation and Allergy-Drug Targets, 8(3):182-190], as well as non-inflammatory arthropathies such as chrondrocalcinosis, and in acute lung injury, sepsis, neuropathic pain and ischemia-reperfusion injury, to name a few. Increased levels of PAF are found in patients with asthma, acute respiratory distress syndrome (ARDS), hydrostatic pulmonary edema, trauma, sepsis and intestinal ischemia reperfusion. Uhlig and Engel (2005) Pharmacol. Reports 57:suppl 206-221.

G. Articles of Manufacture

In another embodiment of the invention, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the anti-PAF antibody. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

H. Structure-based Design of Humanized Monoclonal Antibodies to Recognize Bioactive Lipids: Platform for Drug Discovery

Lpath's proprietary Immune Y2™ technology allows the generation of monoclonal antibodies against bioactive lipids, including PAF. Lpath's mAbs Sonepcizumab and Lpathomab (also referred to as LT1009 and LT3015, targeted to S1P and LPA, respectively) are first-in-class examples of antibody drugs against bioactive lipids. Because of similarities in the structural framework of LT1009 and LT3015, and aided by recently derived x-ray diffraction data on LT1009 Fab fragment-S1P co-crystals, it is believed that in silico modeling can be used to generate new mAbs against different bioactive lipid targets, including PAF, without the need to immunize mice. As described below, structure activity relationship (SAR) assays can be used to make mutations in the humanized framework and CDRs of existing humanized monoclonal antibodies to bioactive lipids, such as LT3015 and/or LT1009, to alter their affinity and/or specificity for their respective ligands to produce that bind PAF. With such antibodies in hand, variants that are specifically reactive to PAF and contain at least one CDR peptide having an amino acid sequence that has a sequence identity of at least 65 percent, optionally a sequence identity of at least 80 percent, at least 90 percent, at least 95 percent, and 100 percent identity with an amino acid sequence set forth below can be produced.

The invention will be better understood by reference to the following Examples, which are intended to merely illustrate the best mode now known for practicing the invention. The scope of the invention is not to be considered limited thereto.

### EXAMPLES

### Example 1

### In Silico Design of Anti-Lipid Antibodies

Using computational and structure-based methodology, it is now possible to develop novel therapeutic antibodies that specifically recognize bioactive lipids with high affinity. As a representative example, this approach is applied towards design of an antibody that binds platelet-activating factor (PAF), an inflammatory mediator whose levels in serum are substantially elevated in patients with anaphylactic shock.

As is known, the humanized monoclonal antibody Sonepcizumab™ (LT1009) neutralizes the bioactive signaling lipid, sphingosine-1-phosphate (S1P). The three-dimensional crystal structure of the Fab fragment of LT1009 in complex with S1P (PBD ID 3I9G) has also been described. This structure was found to present a unique mechanism where divalent metal atoms bridge the antibody-antigen interface. The structure revealed interactions that govern lipid recognition by therapeutic antibodies and identified specific amino acids and functional groups critical for lipid binding.

Based on the Fab-S1P structure, introducing the following amino acids into the light chain of LT1009 was predicted to increase binding of the antibody to PAF: L30K, L31R, L32N, L50Q, L92R, and L93G (see sequence in Table 1 below). Using this information, a light chain variant of LT1009 was designed in silico, and subsequently generated. The variable domain sequence harboring these mutations was synthesized and cloned into a vector containing the light chain constant region of the antibody. The resulting plasmid (pATH334), along with the heavy chain plasmid (pATH221), was purified and transiently transfected into a HEK293 cell culture. Concurrently, an additional culture was transiently tranfected with plasmids encoding the parent light chain (pATH320) and heavy chain (pATH221) genes of LT1009. The amino acid sequences of the parent LT1009 and variant light chain variable regions are shown in Table 1, below.

TABLE 1
Amino acid sequences of LT1009 and PAF-binding variant

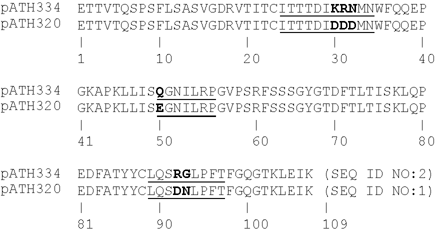

Amino acid sequences of the light chain variable region of LT1009 (pATH320, SEQ ID NO: 1) and of the variant (pATH 334, SEQ ID NO: 2) designed to have enhanced binding to PAF. The six residues in bold differ between the two sequences. All six residues are located with the CDRs (underlined).

As shown above, CDRL1 has the amino acid sequence ITTTDIKRNMN (SEQ ID NO: 3), CDRL2 has the amino acid sequence QGNILRP (SEQ ID NO: 4), and CDRL3 has the amino acid sequence LQSRGLPFT (SEQ ID NO: 5). This antibody is LPT-1009-PAF.

After 5 days in culture, the supernatants were harvested and the antibodies were purified using protein-A affinity chromatography. The affinity of the LT1009 (pATH320×pATH221) and LPT-1009-PAF variant (pATH334×pATH221) antibodies for PAF was measured using a direct binding ELISA. Microtiter ELISA plates were coated with thiolated PAF conjugated to delipidated BSA. Thiolated PAF ([IUPAC name: (R)-2-acetoxy-3-((16-mercaptohexadecyl)oxy)propyl(2-(trimethylammonio)ethyl) phosphate; alternatively this can be named using lipid nomenclature: 1-(16-mercaptohexadecyl)-2-acetoyl-/sn/-glycero-3-phosphocholine] and thiolated PAF-BSA conjugates were prepared as for thiolated S1P and thiolated S1P-BSA conjugates, as described hereinabove and in, for example, commonly owned U.S. patent application Ser. No. 11/755,352 (publication no. 20070281320), which is incorporated herein in its entirety for all purposes.

For the ELISA, either the LT1009 or the LPT-1009-PAF variant antibody was titrated and incubated for 1 hour. The plates were extensively washed and the bound antibodies were detected with HRP conjugated goat anti-human (H+L) secondary antibody and developed with tetramethyl-benzidine substrate using standard methods. The optical density (OD) was measured at 450 nm using a Thermo Multiskan EX.

The mutations introduced into LT1009 caused a dramatic effect on the ability of the antibody to bind PAF. While the LT1009 antibody (pATH320×pATH221) has no measurable binding affinity to PAF-BSA conjugate in the assay, the variant antibody (pATH334×pATH221) showed a saturated binding isotherm with an EC₅₀ of approximately 2 nM.

Thus when combined with the LT1009 heavy chain, the variant light chain containing six mutations that were predicted to increase binding to PAF, yielded an antibody that bound PAF with high affinity. In contrast, the LT1009 antibody showed no detectable PAF binding. Furthermore, while the variant antibody retains some binding for S1P, this is greatly decreased from the S1P binding affinity of LT1009. This high affinity binding by the variant antibody demonstrates that the antigen specificity of anti-lipid antibodies can be modulated using structural modeling and computational approaches. This demonstrates the successful in silico design of a novel antibody with desired characteristics.

Anti-PAF antibody variants according to the invention will specifically bind PAF and comprise at least one, e.g. from about one to about ten, and preferably from about two to about five, substitutions in one or more CDR regions of the parent antibody, here an anti-PAF antibody having a light chain variable regions amino acid sequence of pATH 334. Ordinarily, a variant will have an amino acid sequence having at least 50% amino acid sequence identity with the parent antibody heavy or light chain variable domain sequences, more preferably at least 65%, more preferably at 80%, more preferably at least 85%, more preferably at least 90%, and most preferably at least 95%. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind PAF and preferably has desired activities which are superior to those of the parent antibody. For example, the variant may have a stronger binding affinity, different pharmacokinetic or toxicological properties, or enhanced ability to modulate inflammation. To analyze such desired properties (for example les immunogenic, longer half-life, enhanced stability, enhanced potency), one should compare a Fab form of the variant to a Fab form of the parent antibody or a full length form of the variant to a full length form of the parent antibody.

### Example 2

### Generation of Monoclonal Antibodies to PAF Using the Immune Y2 Method

Preparation of Thiolated PAF:

The synthetic approach described in this example results in the preparation of thiolated PAF. The derivatized PAF can then be further complexed to a carrier, for example, a protein carrier, which can then be administered to an animal to elicit an immunogenic response to PAF.

General Procedure

Thin layer chromatography was performed on 0.25 mm pre-coated glass plates (Merck silica gel 60F₂₅₄) and detection by ammonium molybdate stain. Chromatography was performed using an Isco CombiFlash Companion system suing standard columns (Silicycle). Flash column chromatography (FCC) used silica gel 60 (230-400 mesh, Silicycle). All reagents were purchased from Aldrich, TCI, or Acros and used without further purification. 16-bromohexadecanol was purchased from Astatech Inc. Dry solvents were purchased from Acros (AcroSeal®). ¹H (400 MHz), and ³¹P (162 MHz) NMR spectra were recorded at 25° C. on a Varian INOVA instrument. Chemical Shifts are given in ppm. Mass spectra were measured at the University of Utah Medicinal Chemistry Department using either matrix assisted laser desorption ionization (MALDI) or electrospray ionization (ESI).

16-(tert-butylsulfanyl)hexadecan-1-ol (1). A solution of n-butyllithium in hexanes (1.6M, 29 mL, 46.4 mmol) was added dropwise to a solution of tert-butylthiol in dry THF (50 mL) under Ar. After 20 min., a solution of 16-bromohexadecanol (4.9 g, 15.2 mmol) in dry THF (40 mL) was added and the reaction stirred overnight at room temperature. Diluted with sat'd NH₄Cl and extracted with EtOAc (3×100 mL). The combined extracts were washed with water then brine, dried (MgSO₄), and evaporated under reduced pressure. Rf=0.29 (3:1 Hex:EtOAc). Yield 5.0 g (100%). ¹H NMR (CDCl₃) 3.60 (2H, t, J=6.8 Hz), 2.48 (2H, t, J=7.6 Hz), 1.53 (4H, m), 1.29 (9H, s), 1.20-1.40 (24H, m).

16-(tert-butylsulfanyl)hexadecyl methanesulfonate (2). Methanesulfonyl chloride (1.5 mL, 19.4 mmol) was added dropwise to a solution of 1 (5.0 g, 15.2 mmol) and triethylamine (3.2 mL, 22.8 mmol) in dry CH₂Cl₂ (80 mL) cooled in an ice-bath. The reaction was allowed to warm to room temperature overnight. The reaction mixture was diluted with CH₂Cl₂, washed with water (2×50 mL) then brine, dried (MgSO₄), and evaporated under reduced pressure. Rf=0.25 (4:1 Hex:EtOAc). Yield 6.2 g (100%). ¹H NMR (CDCl₃) 4.21 (2H, t, J=6.8 Hz), 2.91 (3H, s), 2.52 (2H, t, J=7.6 Hz), 1.75 (2H, m) 1.58 (2H, m), 1.32 (9H, s), 1.20-1.45 (24H, m).

(2S)-3-{[16-(tert-butylsulfanyl)hexadecyl]oxy}propane-1,2-diol (3). Sodium hydride (60% dispersion in mineral oil, 2.0 g, 50 mmol) was added to a solution of 2 (5.8 g, 14.2 mmol) in dry toluene (80 mL) under Ar. A solution of (R)-( )-2,2-dimethyl-1,3-dioxolane-4-methanol (2.8 g, 21.2 mmol) in dry toluene (30 mL) was added and the reaction was heated to reflux for 2 hours. The reaction was cooled to room temp. and sat'd NH₄Cl was added. The layers were separated and the aqueous layer was extracted with EtOAc (2×50 mL).). The combined extracts were washed with water then brine, dried (MgSO₄), and evaporated under reduced pressure. The crude product was suspended in MeOH (60 mL) with p-toluenesulfonic acid (0.23 g, 1.2 mmol) and stirred at room temp. until the reaction was complete by TLC. Triethylamine (1 mL) was added and the solvents were evaporated under reduced pressure. The product was purified by chromatography (0-10% MeOH in CH₂Cl₂). Rf=0.20 (2:3 Hex:EtOAc). Yield: 3.4 g (61%). ¹H NMR (CDCl₃) 3.86 (1H, m), 3.60-3.75 (2H, m), 3.41-3.56 (4H, m), 2.68 (1H, d, J=4.8 Hz), 2.52 (2H, t, J=7.2 Hz), 2.28 (1H, t, J=6.4 Hz), 1.52-1.63 (4H, m), 1.43 (9H, s), 1.24-1.45 (24H, m).

(6R)-2,2,26,26-tetramethyl-3,3-diphenyl-4,8-dioxa-25-thia-3-silaheptacosan-6-ol (4). tert-Butyldiphenylchlorosilane (3 mL, 11.5 mmol) was added dropwise over 10 min. to a solution of 3 (3.4 g, 8.3 mmol) and DMAP (1.51 g, 12.3 mmol) in dry CH₂Cl₂ (290 mL) cooled in an ice-bath under Ar. After 1 min., the reaction was allowed to warm to room temp. and stirred overnight. The solvent was evaporated and the product purified by chromatography (0-15% EtOAc in Hex). Rf=0.43 (4:1 Hex:EtOAc). Yield: 4.7 g (89%). ¹H NMR (CDCl₃) 7.66 (2H, dd, J=1.6, 7.6 Hz), 7.34-7.46 (m, 6H), 3.89 (1H, m), 3.70 (2H, d, J=5.6 Hz), 3.47-3.54 (2H, m), 3.43 (2H, t, J=7.2 Hz), 2.52 (2H, t, J=7.6 Hz), 2.481 (1, m), 1.56 94H, m), 1.32 (9H, s), 1.21-1.42 (24H, m), 1.06 (9H, s).

(6R)-2,2,26,26-tetramethyl-6-(oxan-2-yloxy)-3,3-diphenyl-4,8-dioxa-25-thia-3-silaheptacosane (5). Pyridinium p-toluenesulfonate (73 mg, 0.29 mmol) was added to a solution of 4 (4.7 g, 7.3 mmol) and 2,4-dihydropyran (1.3 mL, 14.6 mmol) in dry CH₂Cl₂ (40 mL) under Ar. The reaction was stirred overnight. The reaction was diluted with CH₂Cl₂ (300 mL), washed with sat'd NaHCO₃, water, then brine, dried (MgSO₄), and evaporated under reduced pressure. The crude product was used directly for the next step.

(2S)-3-{[16-(tert-butylsulfanyl)hexadecyl]oxy}-2-(oxan-2-yloxy)propan-1-ol (6). Glacial acetic acid (520 uL, 9.1 mmol) was added to a solution of 5 (5.3 g, 7.3 mmol) in dry THF (30 mL) under Ar followed by a solution of tetrabutylammonium fluoride in THF (1M, 8.8 mL, 8.8 mmol). After 3 hours additional AcOH (125 mL, 2.2 mmol) and TBAF (2.2 mL, 2.2 mmol) were added and the reaction stirred overnight. The solvent was evaporated under reduced pressure. The residue was dissolved in EtOAc, filtered and then filtrate evaporated under reduced pressure. Ht product was purified by chromatography (15-45% EtOAc in Hex). Rf=0.13 (4:1 Hex:EtOAc). Yield 2.4 g (69%, 2 diastereomers). ¹H NMR (CDCl₃) 4.76 (0.5H, m), 4.58 (0.5H, m), 3.68-4.02 (4H, m), 3.38-3.60 (m, 6H), 2.52 (2H, t, J=7.2 Hz), 1.43-1.90 (10H, m), 1.32 (9H, s), 1.15-1.41 (27H, m).

3-{[(2-bromoethoxy)(diisopropylamino)phosphanyl]oxy}propanenitrile (7). 2-Bromoethanol (1.1 mL, 15.4 mmol) was added dropwise to a solution of 2-cyanoethyl N,N-diisopropylchlorophosphoramidite (5.0 g, 16.6 mmol) and tetrazole (540 mg, 7.7 mmol) in dry CH₂Cl₂ (100 mL). After stirring g for 1 hour, the solvent was evaporated under reduced pressure and the product purified by FCC (17:3:1 Hex:EtOAc:NEt₃). Rf=0.66 (4:1 Hex:EtOAc). Yield 3.9 g (86%). ¹H NMR (CDCl₃) 3.68-4.02 (4H, m), 3.55-3.68 (2H, m), 3.50 (2H, t J=6.4 Hz), 2.66 (2H, td, J=5.6, 0.8 Hz), 1.20 (12H, dd, J=6.4, 2.0 Hz); ³¹P NMR (CDCl₃) 149.91.

3-{[(2-bromoethoxy)[(2R)-3-{[16-(tert-butylsulfanyl)hexadecyl]oxy}-2-(oxan-2-yloxy)propoxy]phosphoryl]oxy}propanenitrile (8). A solution of 7 (1.46 g, 5.0 mmol) in dry CH₂Cl₂ (30 mL) was added to a solution of 6 (1.73 g, 3.5 mmol) in dry CH₂Cl₂ (20 mL) under Ar. The reaction was stirred for 2 hours at room temp. A solution of iodine (1.26 g, 5.0 mmol) in 7:2:2 THF:pyridine:water was added dropwise over 30 minutes. The reaction was diluted with CH₂Cl₂ and poured in to a solution of sat'd Na₂S₂O₃. The layers were separated and the aqueous phase was extracted with CH₂Cl₂ (2×). The combined extracts were washed with 2M HCl, water then brine, dried (MgSO₄), and evaporated under reduced pressure. The product was purified by chromatography (30-85% EtOAc in Hex). Rf=0.19 (2:3 Hex:EtOAc). Yield 2.4 g (93%). ¹H NMR (CDCl₃) 4.78 (1H, m), 4.08-4.41 (6H, m), 3.99-4.05 (1H, m), 3.88-3.97 (1H, m), 3.38-3.64 (7H), 2.75-2.82 (2H, m), 2.52 (2H, t, J=7.2 Hz), 1.48-1.86 (10H, m), 1.32 (9H, s), 1.22-1.43 (24, m); ³¹P NMR (CDCl₃) −0.86, −1.00, −1.03 (2:1:1).

[(2R)-3-{[16-(tert-butylsulfanyl)hexadecyl]oxy}-2-hydroxypropoxy][2-(trimethylaminio)ethoxy]phosphinic acid (9). Gaseous trimethylamine (60 mL) was condensed in a 500 mL pressure vessel cooled to −78° C. A solution of 8 in dry CH₃CN (70 mL) was added via cannula under Ar. The vessel was sealed and heated to 65° C. for 40 hours. The reaction was cooled in an ice bath and the vessel was opened and the solvent was evaporated under reduced pressure. The crude product was dissolved in MeOH and stirred with DOWEX 50×8-100 (H⁺) resin (1.3 g, previously washed with MeOH) for 1 hour. The resin was filtered off and the filtrate was evaporated under reduced pressure. The product was purified by chromatography (5-100% MeOH in CH₂Cl₂). Rf=0.087 (65:35:4 CH₂Cl₂:MeOH:H₂O). Yield 1.5 g (80%). ¹H NMR (CDCl₃) 4.28 (2H, m), 3.81-4.15 (3H, m), 3.65 (2H, m), 3.37-3.47 (4H, m), 3.23 (9H, m), 2.52 (2H, t J=7.2 Hz), 1.48-1.61 (4H, m), 1.32 (9H, s), 1.21-1.44 (m, 24H); ³¹P NMR (CDCl₃) 1.32.

Thio-PAF ([(2R)-2-(acetyloxy)-3-[(16-sulfanylhexadecyl)oxy]propoxy][2-(trimethylaminio)ethoxy]phosphinic acid). Acetic anhydride (0.37 mL 3.9 mmol) was added to a suspension of 9 (1.50 g, 2.6 mmol) and DMAP (150 mg, 1.2 mmol) in dry CH₂Cl₂ (25 mL) and pyridine (20 mL) under Ar. After 3 hours, additional DMAP (150 mg) and Ac₂O (0.4 mL) were added. After 5 hours, additional Ac₂O (0.3 mL) and CH₂Cl₂ (10 mL) were added and the reaction was stirred overnight. The solvent was evaporated under reduced pressure and the residue was co-evaporated with toluene. The crude product was dissolved in dry THF (15 mL) and AcOH (15 mL) under Ar. 2-Nitrophenylsulfenyl chloride (0.74 g, 3.9 mmol) was added and the reaction was stirred for 2-3 hours. H₂O (1 mL) was added followed by a solution of PMe₃ in THF (1M, 5.9 mL, 5.9 mmol). The reaction was stirred for 2 hours then the solvents were evaporated. The product was purified by chromatography (10-100% MeOH in CH₂Cl₂). Rf=0.10 (65:35:4 CH₂Cl₂:MeOH:H₂O). Yield 1.01 g (70%). ¹H NMR (CDCl₃) 5.14 (1H, m), 4.26-4.34 (2H, m), 3.88-4.02 (2H, m), 3.75-3.25 (2H, m), 3.89-4.02 (2H, m), 3.39-3.47 (2H, m), 3.37 (9H, s), 2.52 (2H, dt, J=6.8, 7.6 Hz), 2.07 (3H, s), 1.56-1.64 (2H, m), 1.46-1.56 (2H, m), 1.33 (1H, t, J=7.6 Hz), 1.22-1.44 (24H, m); ³¹P NMR (CDCl₃) 0.49; MALDI-MS: 556.3 (M+H)⁺.

Thio-PAF (16:0) is shown below:

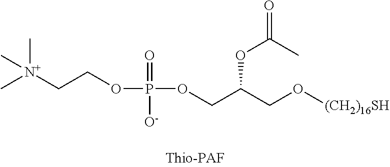

Once the PAF has been thiolated, standard cross-linkers such as succinimidyl 4-[N-maleimidomethyl]cyclohexane-1-carboxylate (SMCC) or N-Succinimidyl[4-iodoacetyl]aminobenzoate (SIAB) may be used to couple a desired carrier protein to the SH group on the lipid. Such protein conjugates are more immunogenic than the lipid alone.

Mice were immunized with PAF-SMCC-OVA (ovalbumin) to obtain immune responses. Four groups of five mice each were immunized for the first time with 100 ul of 100 μg PAF-SMCC-OVA in Complete Freunds adjuvant (C.F.A.) given subcutaneously at two sites. A second injection of 100 μg administered i.p. with incomplete Freunds adjuvant and one final i.p. boost with 10 μg i.p. in Hanks Balanced Salt Solution (HBSS) yielded 9B7. Another group which yielded antibodies 6C5 and 15B3 received a similar initial injection of 100 μg PAF-SMCC-OVA s.c. and only one boost of 10 μg i.p. of the same complex in HBSS.

All antibodies obtained were produced by harvesting spleens with the highest titers as monitored by ELISA on plates coated with PAF-IAB-BSA. The spleens were processed and fused according to standard techniques developed by Kohler and Milstein (1975), Nature, vol. 256:495-497. Screening at this point with a different PAF conjugate (differing in both linker and carrier protein compared to the immunogen) ensures that the antibodies bind specifically to PAF rather than to the SMCC linker or OVA carrier protein in the immunogen.

### Example 3

### Screening of Hybridomas by Direct ELISA

ELISA plates (Corning Costar; Lowell, Mass.) were coated with PAF-SMCC-BSA diluted in 0.1M carbonate buffer (pH 9.5) at 37° C. for 1 h. Plates were washed with PBS (137 mM NaCl, 2.68 mM KCl, 10.1 mM Na₂HPO₄, 1.76 mM KH₂PO₄, pH 7.4) and blocked with 1% BSA in PBS containing 0.1% Tween-20 for 1 hr at room temp or overnight at 4° C. For the primary incubation (1 hr at RT), a dilution series of the anti-PAF mAbs produced from each hybridoma (0.4 μg/mL, 0.2 μg/mL, 0.1 μg/mL, 0.05 μg/mL, 0.0125 μg/mL, and 0 μg/mL) was added to the plate (100 μL/well). Plates were washed and incubated with 100 μL/well HRP-conjugated goat anti-mouse IgG (1:20,000 dilution; cat#115-035-062, Jackson ImmunoResearch,) for 1 hr at RT. After washing, the peroxidase chromogenic substrate, tetramethylbenzidine, (Sigma-Aldrich; St. Louis Mo.) was added, and color development was stopped by adding 1M H₂SO₄. OD values were measured at 450 nm using a Thermo Multiskan EX. Raw data were transferred to GraphPad software for analysis.

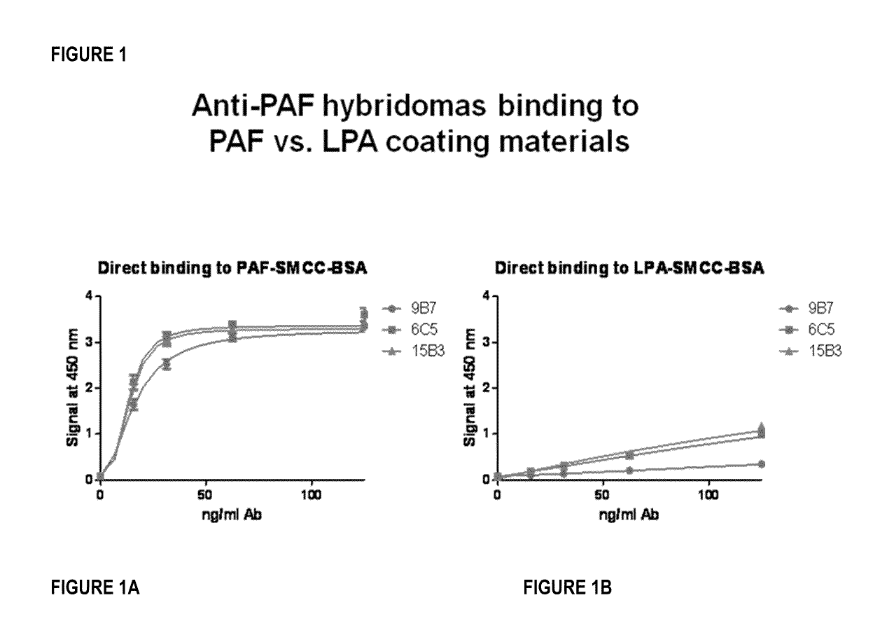

Three antibodies were identified that recognize PAF (PAF-SMCC-BSA or PAF-IAB-BSA coating or “laydown” material) in the direct ELISA, and appear to exhibit similar apparent binding properties for PAF (FIG. 1A). These antibodies do not recognize LPA (FIG. 1B), another bioactive lipid that is structurally very similar to PAF.

### Example 4

### PAF-Binding ELISAs

Biotinylated PAF was prepared by coupling biotin to a thiolated PAF derivative, 1-(16-mercaptohexadecyl)-2-acetoyl-/sn/-glycero-3-phosphocholine (Echelon), using thiol-reactive maleimide chemistry. In separate vials, maleimide-(PEG)₂-biotin (Pierce) and the PAF derivative were dissolved in DMSO to final concentrations of 100 mM and 5 mM, respectively, by sonication and vortex mixing until both solution were clear and particulate-free. Equal volume aliquots of each solution were added to 3-fold excess 1× phosphate buffered saline, pH 7.4 (PBS) and incubated 4 hours at 25° C. The final concentration of biotinylated PAF was assumed to be 1 mM.

Direct binding to biotinylated PAF (PAF-biotin) was measured using an enzyme-linked immunosorbent assay (ELISA) as follows. Fc specific anti-mouse IgG (Jackson Immunoresearch) was diluted to 1 μg/mL in 0.1 M carbonate buffer pH 9.5 and 15 μL/well was used to coat 384 well plates (Greiner) overnight at 4° C. Each well was blocked by adding 25 μL of PBS, 0.01% Tween-20 containing 1% BSA (blocking buffer) and incubated for 1 hour at room temperature followed by 3 washes with PBS. Anti-PAF antibody samples were diluted to 50 ng/mL with blocking buffer, loaded onto the plate (15 μL/well), and incubated for 1 hour at room temperature and washed 3 times with PBS. Two-fold serial dilutions of PAF-biotin were prepared in blocking buffer and 100 μL were added to the captured antibody and incubated for 4 hours. The unbound lipid was removed by washing the plate 3 times with PBS. The antibody-bound PAF was detected by adding 15 μL of 1:60,000 dilution of horseradish peroxidase (HRP) conjugated streptavidin (Jackson Immunoresearch), incubating for 15 minutes, washing 3 times with PBS, and adding 15 μL of cold tetramethylbenzidine substrate (Sigma), and quenching by the addition of 1 M H2504. The optical density (OD) was measured at 450 nm using a Perkin-Elmer Victor3 plate reader and the data was plotted using Graphpad Prism software.

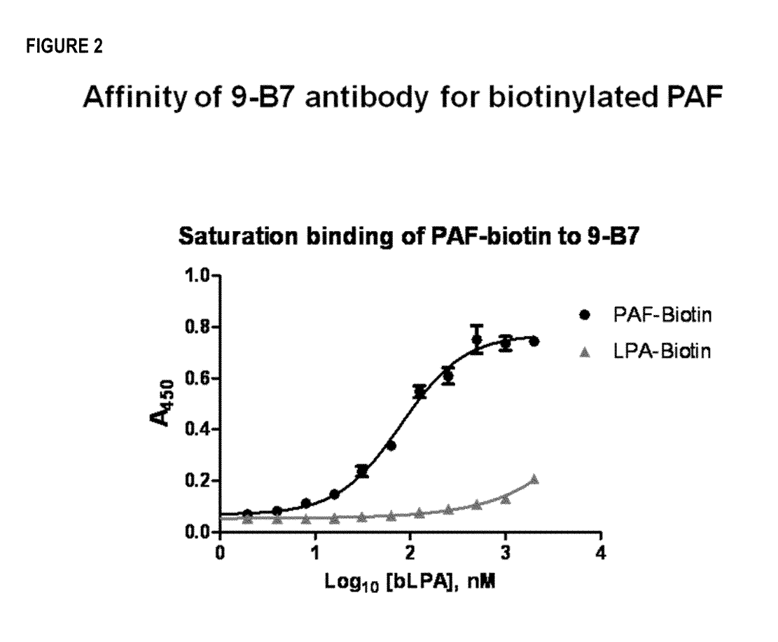

Using this “inverted ELISA,” saturation binding of PAF-biotin to antibody 9-B7 was determined. As shown in FIG. 2, the EC50 for binding of this antibody to the labelled PAF is approximately 200 nM, and is negligible for binding of this antibody to biotinylated LPA.

### Example 5

### Anti-PAF Antibodies Block PAF Signalling in DiscoveRx PAF Receptor Signaling Assay

The ability of the murine anti-PAF antibodies to block PAF signalling via its receptor was studied in a series of assays using cells supplied by DiscoveRx (Freemont, Calif.). The DiscoveRx PathHunter CHO-K1 PATFR β-arrestin cell line was used to test PAF signalling in cells overexpressing the PAF GPCR.

Cells

This assay is based on a CHO-K1 PATFR-Arrestin clone (PathHunter™, cat. 93-0236E2) sold by DiscoveRx Corporation (42501 Albrae St., Freemont, Calif. 94538).

PathHunter™ Cell-Based Assay

The PathHunter™ products use Enzyme Fragment Complementation technology in which two weakly complementing fragments of the B-galactosidase (-gal) enzyme are expressed within stable transfected cells. In this system, one fragment of the -gal, termed the enzyme acceptor (EA), is fused to the C-terminus of the -arrestin2. The complementing fragment of -gal, termed the ProLink™ tag, is expressed as a fusion protein with the PAF receptor at the C-terminus. Upon activation, the PAF₁ receptor is phosphorylated, providing a binding site for -arrestin. The interaction of -arrestin and the PAF receptor forces the interaction of Prolink and EA, thus allowing complementation of the two fragments of -gal and the formation of functional enzyme capable of hydrolyzing substrate and generating a chemiluminescent signal. Complementation is driven by protein-protein interaction between arrestin-EA and ProLink-labeled PAF Receptor.

Thawing and Maintenance of Cells

PTAFR cells were expanded following the manufacturer's recommendations. Briefly, a frozen vial of cells was thawed in a 37° C. water bath under sterile conditions until just before ice completely melted. DMSO was removed by transferring the cells to a sterile 50 mL conical tube with 15 mL of pre-warmed complete medium without antibiotics [F12 nutrient mix with (+) L-glutamine, Gibco Cat. 10378, supplemented with 10% fetal bovine serum (Hyclone Cat. SV0014.03)] and then centrifuged at 1100 rpm for 5 minutes. The supernatant was removed and the cell pellet re-suspended in 5 mL of pre-warmed complete medium without antibiotics and transferred to T150 flask with 25 mL of complete medium without antibiotics. The flask was incubated at 37° C. in a humidified 5% CO₂ atmosphere. After 24 hours, the media was exchanged with 30 mL of complete growth medium with antibiotics (F12 nutrient mix supplemented with 10% fetal bovine serum, 100 U/mL penicillin, 100 μg/mL streptomycin, 292 μg/m L-glutamine, Gibco, cat. 10378, 300 μg/mL hygromycin, Invitrogen cat. 10687-010, and 800 μg/mL G418, Omega Scientific GN-04). Cells were passaged every 2-3 days at a 1:6 dilution in complete growth media with antibiotics using a 0.05% trypsin solution (Cellgro cat. 25-053 Cl). Cells were maintained at 70% confluence and not allowed to grow at confluence for more than 24 hours.

Plating Cells

To prepare assay plates, cells were collected into a sterile 50 mL conical tube using 5 mL of a non-enzymatic cell dissociation buffer (CellStripper, Cellgro cat. 25-056 Cl). Cells were spun at 1100 rpm for 5 minutes, supernatant removed, and the cell pellet was re-suspended in 5 mL of complete medium with antibiotics. Then the cells were counted using an automated cell counter and plated at 10,000 cells/well (100 μL total volume in each well) in 96-well white clear-bottom plates. Plates were incubated at 37° C. in a humidified 5% CO₂ atmosphere. After 24 hours, plates were starved with OPTI-MEM (reduced serum medium with HEPES, 2.4 g/L sodium bicarbonate, and L-glutamine, Gibco cat. 31985). This was done by quickly and gently dumping the plate upside down to remove medium and then adding 100 μL of OPTI-MEM to each well. Plates were incubated at 37° C. for another 24 hours.

Treatment of Cells

A 100 μM stock of PAF (Avanti cat. 840009) was prepared according to the vendor's instructions. The stock PAF was diluted into delipidized human serum (DHS, Biocell cat. 1131-00) to achieve a final concentration of 5 nM PAF in each well of a 96 deep well 2.0 mL plates. For inhibition of PAF signaling, 5 nM PAF was incubated with increasing amounts (0 to 0.5 mg/mL) of the anti-PAF antibody. The medium from a 96-well plate containing the cells was removed by quickly but gently dumping the plate upside down, ensuring that the plate remained parallel to the table, and 100 μL of prepared standard or sample (in duplicate) was added to each well. The plates were incubated at 37° C. in a humidified 5% CO₂ atmosphere for 90 minutes. Plates were then washed with 300 μL of OPTI-MEM. After removal of this wash medium by dumping the plate upside down, 100 μL of OPTI-MEM was added to each well. 25 μL of freshly made working detection reagent solution (Cell Assay Buffer, Emerald II, and Galacton Star mixed in a ratio of 19:5:1, respectively, DiscoveRx cat. 93-001) was added to each well. The plates were incubated in the dark at room temperature for an additional 90 minutes. Finally, the plates were read on a standard luminescence plate reader. The ability of anti-PAF antibodies to decrease signaling in response to 5 nM PAF in this assay was measured. The signal (in relative light units, or ‘RLUs’) was graphed for each antibody using a four-parameter fit equation (GraphPad Prism5 software) to calculate the potency (IC50).

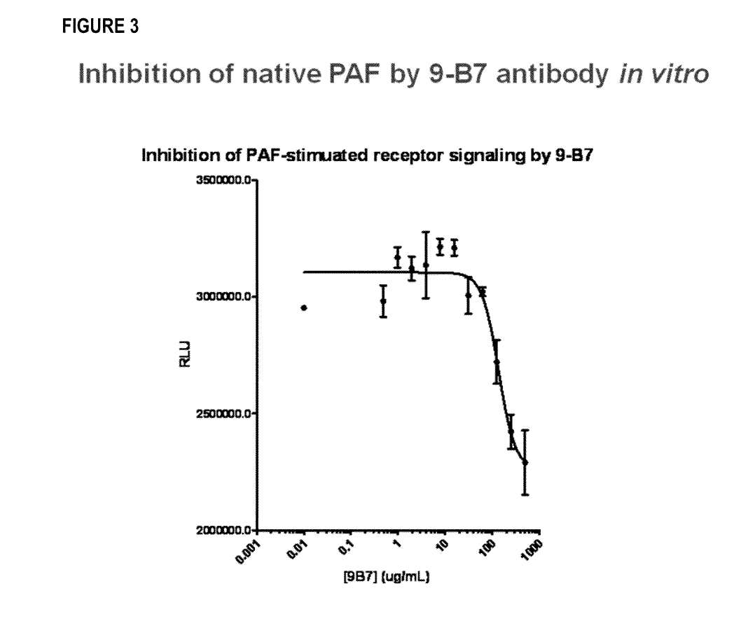

FIG. 3 shows that anti-PAF antibody 9B7 inhibits PAF-stimulated receptor signaling, indicating that this antibody binds to native PAF and interferes with its biological effects in a cellular environment.

All of the compositions and methods described and claimed herein can be made and executed without undue experimentation in light of the present disclosure. While the compositions and methods of this invention have been described in terms of preferred embodiments, it will be apparent to those of skill in the art that variations may be applied to the compositions and methods. All such similar substitutes and modifications apparent to those skilled in the art are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. An isolated monoclonal antibody, or an antigen binding fragment thereof, that binds platelet activating factor (PAF), wherein the isolated antibody or antigen binding fragment thereof optionally comprises at least one heavy chain variable region and at least one light chain variable region, wherein each light chain variable region comprises at least one of the following light chain CDRs:
(i) a CDRL1 comprising the amino acid sequence ITTTDIKRNMN (SEQ ID NO: 3) or a variant thereof in which from 1 to 10 amino acids residues are substituted with a different amino acid residue;
(ii) a CDRL2 comprising the amino acid sequence QGNILRP (SEQ ID NO: 4) or a variant thereof in which from 1 to 6 amino acids residues are substituted with a different amino acid residue; and
(iii) a CDRL3 comprising the amino acid sequence LQSRGLPFT (SEQ ID NO: 5) or a variant thereof in which from 1 to 8 amino acids residues are substituted with a different amino acid residue.

2. An isolated antibody or antigen binding fragment of claim 1 that comprises a light chain variable domain comprising a sequence of amino acid residues having an amino acid sequence selected from the group consisting of: a.
(SEQ ID NO: 2)
ETTVTQSPSFLSASVGDRVTITCITTTDIKRNMNWFQQEPGKAPKLLI
SQGNILRPGVPSRFSSSGYGTDFTLTISKLQPEDFATYYCLQSRGLPF
TFGQGTKLEIK;
and
b. a sequence of amino acid residues having an amino acid sequence that has at least 50%, 65%, 80%, 85%, 90%, or 95% sequence identity with the amino acid sequence:
(SEQ ID NO: 2)
ETTVTQSPSFLSASVGDRVTITCITTTDIKRNMNWFQQEPGKAPKLLI
SQGNILRPGVPSRFSSSGYGTDFTLTISKLQPEDFATYYCLQSRGLPF
TFGQGTKLEIK.

3. An isolated antibody or antigen binding fragment according to claim 1 wherein at least one amino acid residue of the antibody or antigen binding fragment is glycosylated.

4. An isolated humanized antibody according to claim 1 that comprises two heavy chains and two light chains.

5. A pharmaceutical composition comprising an isolated antibody or antigen binding fragment according to claim 1 and a pharmaceutically acceptable carrier.

6. A method selected from the group consisting of:
a. a method of reducing inflammation, allergic response or immune response in a subject, comprising administering to a subject having undesired inflammation, allergic response or immune response a therapeutically effective amount of an isolated antibody, or an antigen binding fragment thereof, according to claim 1; and
b. a method of treating a disease or condition in a subject, comprising administering to a subject a therapeutically effective amount of an isolated antibody, or an antigen binding fragment thereof, according to claim 1, wherein the disease or condition is an inflammatory diseases or condition, a disease or condition having an inflammatory component, an autoimmune disease or condition, an allergic condition, inflammatory bowel disease, ulcerative colitis, Crohn's disease, spondyloarthropathy, osteoarthritis, rheumatoid arthritis, multiple sclerosis, immune suppression, systemic lupus erythematosis, psoriasis, asthma, glomerulonephritis, thyroiditis, chondrocalcinosis, acute lung injury, sepsis, ischemia-reperfusion injury, acute respiratory distress syndrome, neuropathic pain, hydrostatic pulmonary edema or trauma.

7. A diagnostic reagent comprising a derivatized platelet activating factor (PAF), wherein said derivatized PAF comprises a polar head group and at least one hydrocarbon chain, wherein a carbon atom within a hydrocarbon chain is derivatized with a reactive group, wherein the reactive group optionally is a sulfhydryl (thiol) group, and wherein the derivatized PAF optionally is (i) associated with a solid support, wherein the association optionally is a covalent association, or (ii) conjugated to a carrier moiety, wherein the carrier moiety optionally is selected from the group consisting of polyethylene glycol, colloidal gold, adjuvant, a silicone bead, and a protein, wherein the protein is optionally selected from the group consisting of keyhole limpet hemocyanin, albumin, ovalbumin, bovine thyroglobulin, and soybean trypsin inhibitor.

8. An ELISA kit comprising a diagnostic reagent according to claim 7 and an agent that binds PAF under physiological conditions, wherein the agent optionally is an antibody, or an antigen binding fragment thereof, that binds PAF.

9. A method of detecting a platelet binding factor (PAF) binding agent in a biological sample, comprising detecting binding of a PAF binding agent in a biological sample to a diagnostic reagent according to claim 7 under conditions that allow the diagnostic reagent to bind the PAF binding agent, if present in the sample, wherein (i) the PAF binding agent optionally is an antibody, antibody fragment or antibody derivative, and (ii) the biological sample optionally is selected from the group consisting of a tissue sample, optionally a biopsy sample, and a fluid sample, wherein the fluid sample optionally is selected from the group consisting of whole blood, plasma, serum, urine, semen, bile, aqueous humor, vitreous humor, synovial fluid, bronchioalveolar lavage fluid, mucous, and sputum.

10. A method selected from the group consisting of:
a. a method of detecting platelet activating factor (PAF) or a metabolite thereof in a sample, comprising detecting binding of PAF or a metabolite thereof in a sample to an anti-PAF antibody or antigen binding fragment thereof of claim 1 under conditions that allow the anti-PAF antibody to bind to the PAF or metabolite thereof, if present in the sample;
b. a method of detecting in a sample a platelet activating factor (PAF) binding agent comprising contacting a sample with a diagnostic device bearing a diagnostic reagent according to claim 7, wherein the reactive group of the diagnostic reagent optionally is a sulfhydryl (thiol) group and wherein the diagnostic reagent optionally comprises a label, wherein the label optionally is biotin.

11. A method according to claim 10 wherein the sample is a tissue sample, optionally a biopsy sample, and a fluid sample, wherein the fluid sample optionally is selected from the group consisting of whole blood, plasma, serum, urine, semen, bile, aqueous humor, vitreous humor, synovial fluid, bronchioalveolar lavage fluid, mucous, and sputum.
